(19) United States
(12) Patent Application Publication
(10) Pub. No.: US 2023/0028254 A1
(43) Pub. Date: Jan. 26, 2023

(54) SEPARATION MEDIA AND PURIFICATION METHODS FOR NUCLEOTIDES AND NUCLEOTIDE COMPONENTS USING THE SAME

(71) Applicant: Donaldson Company, Inc., Bloomington, MN (US)

(72) Inventors: Jinxiang Zhou, Greenville, SC (US); Graham Temples, Greenville, SC (US); Bin Guo, Greer, SC (US)

(21) Appl. No.: 17/862,364

(22) Filed: Jul. 11, 2022

Related U.S. Application Data
(60) Provisional application No. 63/203,198, filed on Jul. 12, 2021.

(51) Int. Cl. B01D 15/38; B01J 41/16; B01J 39/22; B01D 69/14; B01D 15/36

(52) U.S. Cl. CPC B01D 15/3828; B01J 41/16; B01J 39/22; B01D 69/147; B01D 15/362; B01D 15/363

(57) ABSTRACT

Separation media includes a membrane and a plurality of ligands immobilized on the membrane, the plurality of ligands comprising anion-exchange ligands, cation-exchange ligands, thiophilic ligands, hydrophilic ligands, hydrophobic interaction ligands, or a combination thereof. The separation media may be multimodal. The separation media may be configured for separation of target molecules comprising a nucleic acid, nucleotide, nucleoside, nucleobase, or an analogue or derivative thereof, from a reaction mixture. The separation media may be configured for use with organic solvents. A separation device includes the separation media. Materials including a nucleic acid, nucleotide, nucleoside, nucleobase, or an analogue or derivative thereof, may be purified at high speeds using the separation device.

### PRIORITY APPLICATION(S)

This application claims the benefit of U.S. Provisional Application No. 63/203,198, filed 12 Jul. 2021, the disclosure of which is incorporated by reference herein in its entirety.

### FIELD

The present disclosure relates to separation media useful for separation of biomolecules and ions, such as nucleotides, nucleosides, or nucleobases, from a solution, suspension, or dispersion. The separation media of the present disclosure may be used for separations in membrane chromatography. The present disclosure further relates to methods of making and using the separation media.

### BACKGROUND

The use of nucleotides, nucleosides, and their analogues in therapeutics is a rapidly growing industry segment. As nucleic acid therapeutics are developed and their production upscaled, there is a need for improved separation and purification methods.

### SUMMARY

Separation media useful for separation of target molecules from a solution, suspension, or dispersion is disclosed. The target molecules may be biomolecules or ions, including nucleotides or nucleosides. The separation media of the present disclosure may be used for separations in membrane chromatography. The present disclosure further relates to methods of making and using the separation media.

According to an embodiment a separation media includes a membrane; and a plurality of ligands immobilized on the membrane. The plurality of ligands may include anion-exchange ligands, cation-exchange ligands, thiophilic ligands, hydrophobic interaction ligands, hydrophilic ligands, or a combination thereof. The separation media may be configured for separation of target molecules comprising nucleotides, nucleosides, nucleobases, their derivatives and analogues, and combinations thereof, from a reaction mixture. The separation media may be configured for use with organic solvents.

The plurality of ligands may include anion exchange ligands including an aliphatic diamine or triamine comprising 1 to 18 carbons between adjacent amines. The anion exchange ligand may include N,N-dimethylethylenediamine, N,N-dimethylpropylenediamine, N,N-dimethylpropylenediamine, N,N-diethylpropyllenediamine, or a combination thereof.

The plurality of ligands may include cation-exchange ligands comprising aminocarboxylic acids, aminosulfonic acids, or a combination thereof. The cation-exchange ligand may include aminobenzoic acid, aminodiacetic acid, aminopropanoic acid, 3-amino-1-propanesulfonic acid, 3-amino-1-ethylsulfonic acid, or a combination thereof.

The plurality of ligands may include two or more of anion exchange ligands, cation exchange ligands, thiophilic ligands, hydrophilic ligands, and hydrophobic interaction ligands. The plurality of ligands may include ligands with cation-exchange functionality and thiophilic functionality. The plurality of ligands may include mercaptobenzoic acid, mercaptosulfonic acid, a salt thereof, or a combination thereof. Preferably the plurality of ligands may include sodium 3-mercapto-1-propanesulfonate.

A separation device may include a housing; and separation media disposed within the housing. The separation media may include a membrane and a plurality of ligands immobilized on the membrane, the plurality of ligands comprising anion-exchange ligands, cation-exchange ligands, thiophilic ligands, hydrophobic interaction ligands, hydrophilic ligands, or a combination thereof. The housing may include a cassette or a column. The separation media may be configured for separation of target molecules comprising nucleotide, nucleoside, nucleobase, their derivative or analogue, or a combination thereof, from a reaction mixture. The separation media may be configured for use with organic solvents.

A method of purifying a target molecule may include: passing a solution comprising the target molecule through a membrane chromatography device. The target molecule may include a nucleotide, nucleoside, nucleobase, their derivative or analogue, or a combination thereof. The membrane chromatography device may include a housing; and separation media disposed within the housing. The separation media may include a membrane and a plurality of ligands immobilized on the membrane, the plurality of ligands comprising anion-exchange ligands, cation-exchange ligands, thiophilic ligands, hydrophobic interaction ligands, hydrophilic ligands, or a combination thereof. The housing may include a cassette or a column. The separation media may be configured for separation of target molecules comprising nucleotide, nucleoside, nucleobase, their derivative or analogue, or a combination thereof, from a reaction mixture. The separation media may be configured for use with organic solvents.

The target molecule may be purified from a solution comprising a reaction mixture after synthesis of the nucleotide, nucleoside, nucleobase, their derivative or analogue, or a combination thereof. The solution may include an organic solvent. The residence time of the solution in the membrane chromatography device may be 60 s or lower.

### BRIEF DESCRIPTION OF FIGURES

FIG. 1A is a schematic depiction of separation media according to an embodiment.

FIG. 1B is a schematic perspective view of a separation device containing the separation media of FIG. 1A.

FIG. 2 is a graphical representation of dynamic binding capacity data from Example 3.

FIGS. 3A-3C are graphical representations of data from Example 4.

FIG. 4 is a graphical representation of dynamic binding capacity and pressure data from Example 4.

FIGS. 5A and 5B are graphical representations of bind-and-elute data from Example 5.

FIG. 6A is a chromatogram from Example 6.

FIG. 6B is a graphical representation of DBC_{10%} and recovery data from Example 6.

FIG. 7A is a graphical representation of dynamic binding capacity data from Example 7.

FIG. 7B is a chromatogram from Example 7.

FIG. 8A shows overlaid chromatograms from Example 8.

FIG. 8B is a TLC plate of samples from Example 8.

FIGS. 9A-9C are graphical representations of bind-and-elute data from Example 9.

FIG. 9D is a comparison of chromatograms from Example 9.

FIG. 10 is a graphical representation of data from Example 10.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, isotactic, syndiotactic, and atactic symmetries.

The term “aromatic ring” is used in this disclosure to refer to a conjugated ring system of an organic compound. Aromatic rings may include carbon atoms only, or may include one or more heteroatoms, such as oxygen, nitrogen, or sulfur.

The term “alkylated” is used in this disclosure to describe compounds that are reacted to replace a hydrogen atom or a negative charge of the compound with an alkyl group, such that the alkyl group is covalently bonded to the compound.

The term “alkyl” is used in this disclosure to describe a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 6 carbon atoms, 1 to 4 carbon atoms, or 1 to 3 carbon atoms. Examples of alkyl groups include methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, etc.

The term “nucleic acid” and/or “oligonucleotide” as used herein refers to a polymer containing at least two nucleotides (e.g., deoxyribonucleotides or ribonucleotides) in either single- or double-stranded form and includes DNA and RNA. “Nucleotides” include a sugar, a base (sometimes called a nucleobase), and a linking group. In some embodiments, the sugar may be natural deoxyribose or a natural ribose (e.g., DNA and RNA, respectively). Nucleotides are linked together through the linking group to form oligonucleotides. In some embodiments, the linking group may be a phosphate group. A polymer of covalently bonded linking groups may be termed a backbone. “Nucleoside” is otherwise similar to a nucleotide except that a nucleoside does not include a linking group, such as a phosphate group. “Bases” or “nucleobases” include purines and pyrimidines, which further include natural compounds adenine, thymine, guanine, cytosine, uracil, inosine, and natural analogs, and synthetic derivatives of purines and pyrimidines, which include modifications which place new reactive groups such as amines, alcohols, thiols, carboxylates, and alkyl halides. Nucleotides include modified or analog nucleobases, modified or analog sugars, and/or modified or analog linking groups. The modified nucleobases, modified sugars, and/or modified linking groups may be non-canonical/chemically-modified nucleobases, sugars, and/or linking groups which may be synthetic, naturally occurring, and/or non-naturally occurring, and which have similar binding properties as the reference nucleic acid. Examples of such analogs and/or modified bases, sugars, and/or linking groups include, without limitation, phosphorothioates, phosphoramidates, methyl phosphonates, chiral-methyl phosphonates, 2′-O-methyl ribonucleotides, locked nucleic acids (LNAs), and peptide-nucleic acids (PNAs).

A deoxy-ribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages.

The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases. It should be understood that the terms “polynucleotide” and “oligonucleotide” can also include polymers or oligomers comprising both deoxy and ribonucleotide combinations or variants thereof in combination with backbone modifications, such as those described herein.

The polynucleotides and oligonucleotides described herein may include one or more nucleotide variants, including nonstandard nucleotide(s), non-natural nucleotide(s), nucleotide analog(s), and/or modified nucleotides. Examples of modified nucleotides include diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The polynucleotide or oligonucleotide described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or liking group (e.g., backbone). Backbone modifications can include a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) prevents nuclease recognition and degradation. Backbone modifications can also include peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Curr. Med. Chem., 8 (10): 1157-79, 2001 and Lyer et al., Curr. Opin. Mol. Ther., 1 (3): 344-358, 1999. Nucleic acid molecules described herein may contain a sugar moiety that comprises ribose or deoxyribose, as present in naturally occurring nucleotides, or a modified sugar moiety or sugar analog. Modified sugar moieties include 2′-O-methyl, 2′-O-methoxyethyl, 2′-O-aminoethyl, 2′-Flouro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The methods of the present disclosure encompass separation and/or purification of isolated or substantially purified nucleotides, nucleosides, nucleic acid molecules, and compositions containing those molecules. As used herein, an “isolated” or “substantially purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “kosmotrope” is generally used to denote a solute that increases the degree of ordered-ness of water by stabilizing water-water interactions. Kosmotropes may be ionic or non-ionic. In contrast, the term “chaotrope” is generally used to denote a solute that decreases the degree of ordered-ness of water by destabilizing water-water interactions. Chaotropes may be ionic or non-ionic.

The term “substantially” as used here has the same meaning as “significantly,” and can be understood to modify the term that follows by at least about 90%, at least about 95%, or at least about 98%. The term “substantially free” of a particular compound means that the compositions of the present invention contain less than 1,000 parts per million (ppm) of the recited compound. The term “essentially free” of a particular compound means that the compositions of the present invention contain less than 100 parts per million (ppm) of the recited compound. The term “completely free” of a particular compound means that the compositions of the present invention contain less than 20 parts per billion (ppb) of the recited compound. In the context of the aforementioned phrases, the compositions of the present invention contain less than the aforementioned amount of the compound whether the compound itself is present in unreacted form or has been reacted with one or more other materials.

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

The term “about” is used here in conjunction with numeric values to include normal variations in measurements as expected by persons skilled in the art, and is understood to have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to separation media useful for separation of target molecules from a solution, suspension, or dispersion. The target molecules may be biomolecules or ions, including nucleotide, nucleoside, nucleobase, their derivative or analogue, or a combination thereof. The separation media of the present disclosure may be used for separations in membrane chromatography. The present disclosure further relates to methods of making and using the separation media.

The gene and cell therapy industry has shifted rapidly toward commercial processes due to their promising potential to treat various devastating diseases. Plasmid DNAs (pDNA) are key components in the production of the viral vectors, proteins, and mRNAs that are widely used in gene and cell therapy. There has been a sudden and urgent need for high-capacity, high-quality pDNA production. But pDNA production has become a bottleneck of the industry as the scale-up of pDNA manufacturing is not straightforward. Currently, qualified contract manufacturers have long waiting lists and substantial backlogs to service the high demand. Like many other biologics production schemes, multiple steps and unit operations are involved in pDNA production.

Increasingly, nucleotide, nucleoside, nucleobase, their derivative or analogue, or a combination thereof, are utilized to prepare pharmaceutical compositions. Derivatizations may include, for example, fluorination, sugar replacement, addition of a variety of functional moieties, or a number of other known or new modifications. Such derivatizations may be designed to convert or modify the nucleoside, nucleotide, or nucleobase to key building blocks for nucleic acid therapies, or a more tolerable or effective drug, by improving pharmacokinetics, trafficking, altering the state to a prodrug, or exploiting upregulation of enzymatic pathways endemic to diseased tissue. Such pharmaceutical compositions may be utilized in a variety of contexts including HIV/AIDS treatment and cancer treatment. Such analogues often exploit a pathway upregulated by HIV or cancer producing cells or include analogues that induce a mismatch or other replication/translation error, arrest division, and/or induce cellular death.

Additionally, with the rise in nucleic acid therapeutics, additional substitutions and modifications are being pursued to improve the tolerability of the therapeutic by either improving stability or reducing or upregulating immunogenic properties. For example, modifications to cytidine and uridine bases may include 5-iodocytidine-5′-triphosphate, 5-methylcytidine-5′-triphosphate, 2-thiocytidine-5′-triphosphate, 6-azacytidine-5′-triphosphate, 5-bromocytidine-5′-triphosphate, 5-aminoallylcytidine-5′-triphosphate, pseudoisocytidine-5′-triphosphate, N⁴-methylcytidine-5′-triphosphate, 5-carboxycytidine-5′-triphosphate, 5-formylcytidine-5′-triphosphate, 5-hydroxymethylcytidine-5′-triphosphate, 5-hydroxycytidine-5′-triphosphate, 5-methoxycytidine-5′-triphosphate, thienocytidine-5′-triphosphate, 5-bromo-2′-deoxycytidine-5′-triphosphate, 5-propynyl-2′deoxycytidine-5′-triphosphate, 5-iodo-2′-deoxycytidine-5′-triphosphate, 5-methyl-2′-deoxycytidine-5′-triphosphate, 2′-deoxy-P-nucleoside-5′-triphosphate, 5-hydroxy-2′deoxycytidine-5′-triphosphate, 2-thio-2′-deoxycytidine-5′-triphosphate, 5-aminoallyl-2′-deoxycytidine-5′-triphosphate psudouridine-5′-triphosphate, 2′-O-methylpsudouridine-5′-triphosphate, N¹-methylpseudouridine-5′-triphosphate, N¹-ethylpseudouridine-5′-triphosphate, N¹-methyl-2′-O-methylpseudouridine-5′-triphosphate, N¹-methoxymethylpseudouridine-5′-triphosphate, N¹-propylpseudouridine-5′-triphosphate, or their unphosphorylated bases. Similar modifications can be made for thymidine and guanidine bases.

Traditionally, downstream purification has been expensive, slow, and difficult to scale. Typical nucleotide, nucleoside, nucleobase, their derivative or analogue, or a combination thereof purification trains include various steps, such as filtration, ultra filtration, and various chromatography separations utilizing one or more types of chromatography columns. A typical chromatography column used in nucleotide, nucleoside, nucleobase, or nucleic acid purification may include a packed bed column with a resin configured, for example, for size exclusion chromatography or reverse phase chromatography. Resin based chromatography columns have been the gold standard employed to purify biologics for decades. However, column chromatography in large volumes may be very slow. Resin columns are known to require long residence times to perform adequately.

According to an embodiment, the separation media includes a functionalized substrate. The functionalized substrate maybe a functionalized membrane. In contrast to resin columns, membrane adsorbers perform well at short column residence times, potentially providing rapid separations for biologics. The present disclosure provides membranes that are suitable for separation and purification of various nucleotide, nucleoside, nucleobase, their derivative or analogue, or a combination thereof. Compounds of interest that may be separated using the membranes of the present disclosure are collectively referred to here as target molecules, whether in their charged (ionized) or uncharged state. The target molecules may be present in a solution, suspension, or dispersion. For simplicity, the liquid containing the target molecule is referred to here as a solution. The liquid may be a reaction mixture. For example, the liquid may be the reaction mixture used to prepare or synthesize the target molecules (e.g., nucleotides, nucleosides, nucleobases, their derivatives or analogues, and combinations thereof). The separation media may be configured for purifying the target molecule from the reaction mixture. The separation media may be configured for separating the target molecule from starting materials, intermediates, and other reaction products. The reaction mixture may also include solvents, such as water, organic solvents, or a combination thereof, and soluble components dissolved in the solvent. The separation media may be configured for use with organic solvents. The separation media may be configured to separate or purify the target molecules from a solution comprising organic solvents.

The functionalized substrates of the present disclosure include one or more functional groups that interact with target molecules. In some embodiments, the functional groups have affinity to the target molecules and may either bind to the target molecules or slow down their transfer through or along the membrane.

According to an embodiment, the target molecules include nucleotide, nucleoside, nucleobase, their derivative or analogue, or a combination thereof. Nucleotides and nucleosides include nucleobases as their building blocks. In general, the present disclosure provides membranes for and methods of purifying nucleobases/nucleosides/nucleotides, including natural nucleobases/nucleosides/nucleotides, modified nucleobases/nucleosides/nucleotides, nucleobases/nucleosides/nucleotides analogues, and the like.

The four nucleobases in DNA (adenine (A), cytosine (C), guanine (G), thymine (T)) and the additional base (uracil (U)) have unique properties that can be exploited to enhance the purification train including hydrophobic components, aromatic components, hydrogen bonding donors and receptors, and/or groups that can be induced to have charge. But modification of the nucleobases/nucleosides/nucleotides may alter some of these properties. For example, for separation, compounds with amine groups may be intentionally or inadvertently protected by an agent, such as tert-butyloxycarbonyl protecting group (BOC). Protecting groups are used in synthesis to temporarily mask the characteristic chemistry of a functional group because it interferes with another reaction. Protected amines would be sterically hindered and would have one less hydrogen bond donor site and therefore hindering standard hybridization rules, however it is of note there can be alternative pairing configurations that may or may not be affected by protected amine groups. Often such bases with protected groups are difficult to separate with the traditional chemical separation techniques employed for purification of such nucleobase analogues in a pharmaceutical production setting, such as silica gel chromatography, liquid-liquid extraction, liquid/solid extraction, distillation (often unsafe due to inhalation hazard of common protecting groups).

According to an embodiment the present disclosure provides functionalized substrate (e.g., functionalized membranes) that utilize hybridization and a hybridization-based purification method to purify nucleosides/nucleotides and their analogues and derivatives. For example, single nucleosides, nucleotides, or oligonucleotides may be effectively purified based on methods utilizing hybridization, such as Watson crick base pairing.

In some embodiments, employment of Watson crick base pairing provides highly specific purification of the target molecule. In one embodiment, a nucleotide, a nucleoside, or an oligonucleotide may be immobilized on a substrate to provide a ligand, and may then be utilized to capture its complementary base. For example, adenine (A) or its derivative may be immobilized on the substrate to produce a functionalized substrate. The adenine-functionalized substrate may be used to capture thymine (T), uracil (U), and their derivatives. Thymine (T), uracil (U), or their derivatives may be immobilized to a support to capture adenine (A) and its derivatives. Cytosine (C) may be immobilized to capture guanine (G) and its derivatives, and vice versa.

The substrate used as the base material of the separation media may be any suitable material. In some embodiments, the substrate is or includes a membrane, resin, monolith, hydrogel, woven fibrous substrate, nonwoven fibrous substrate, or a combination thereof. In one embodiment, the functionalized substrate is or includes a membrane. In one embodiment, the functionalized substrate is or includes a woven or nonwoven fibrous substrate. The substrate may be modified to include reactive chemical moieties prior to reaction with the ligand. This may be particularly helpful in the case of non-reactive substrates, such as ePTFE. The modifications may include, for example, plasma treatment, dip coating poly(vinyl alcohol), corona treatment, and the like.

Nonwoven substrates (e.g., webs) are typically created by melt blowing, wet laying, melt spinning, solution spinning, air laying, or electrospinning. Nonwoven webs may additionally be treated through post-processing steps, such as calendaring, embossing, needle-punching, or hydroentangling. Nonwoven substrates may also contain a structural resin that has low binding affinity to biomolecules. Such resins are typically used to increase the strength of nonwoven webs. Many nonwoven substrates contain a mixture of fiber sizes and fiber materials. The fibers used to make the nonwoven and woven substrates may include glass, polypropylene, polyamides, polyesters, cellulosic materials, and the like, and combinations thereof. The fibers may have an average fiber size of 0.1 μm or greater, 1 μm or greater, 2 μm or greater, or 3 μm or greater. The fibers may have an average fiber size of 100 μm or less, 50 μm or less, 25 μm or less, 10 μm or less, or 8 μm or less. Average fiber sizes may range from 0.1 μm to 50 μm, or from 1 μm to 25 μm. The average pore size, measured by capillary flow porometer, may be 1 μm or greater, 2 μm or greater, or 3 μm or greater. The average pore size may be 100 μm or less, 50 μm or less, 25 μm or less, 10 μm or less, or 8 μm or less. The average pore size of suitable nonwoven substrates may range from 0.1 μm to 50 μm, from 1 μm to 10 μm, or from 3 μm to 8 μm. The average pore size of woven substrates may be slightly greater than nonwoven substrates, and may range from 1 μm to 100 μm. The basis weight of the fibrous substrate may be 1 gsm (grams per square meter) or greater, 10 gsm or greater, or 20 gsm or greater. The basis weight of the fibrous substrate may be 200 gsm or less or 80 gsm or less. The basis weight of the fibrous substrate may be in a range of 1 gsm to 200 gsm, or from 20 gsm to 80 gsm.

According to an embodiment, the substrate is or includes a membrane. A membrane is understood as a sheet of material with a continuous pathway of polymeric material in all dimensions. Examples of membrane materials include polyolefins, polyethersulfone, poly(tetrafluoroethylene), nylon, fiberglass, hydrogel, polyvinyl alcohol, natural polymers such as cellulose, cellulose ester, cellulose acetate, regenerated cellulose, cellulosic nanofiber, cellulose derivatives, agarose, chitosan, polyethylene, polyester, polysulfone, expanded polytetrafluoroethylene (ePTFE), polyvinylidene fluoride, polyamide (Nylon), polyacrylonitrile, polycarbonate, and combinations thereof.

According to an embodiment, useful membranes have an average pore size, as measure by a capillary flow porometer, of 10 μm or less, 5 μm or less, 2 μm or less, 1 μm or less, 0.45 μm or less, or 0.2 μm or less. The membrane may have an average pore size of 0.1 μm or greater, 0.2 μm or greater, 0.45 μm or greater, 0.7 μm or greater, or 1 μm or greater. The membrane may have an average pore size ranging from about 0.1 μm to 10.0 μm, 0.1 μm to 0.2 μm, 0.1 μm to 0.45 μm, 0.1 μm to 1 μm, 0.1 μm to 2 μm, 0.2 μm to 0.45, 0.2 μm to 1 μm, 0.2 μm to 2 μm, 0.2 μm to 10 μm, 0.45 μm to 1 μm, 0.45 μm to 2 μm, 0.45 μm to 10 μm, 1 μm to 2 μm, or 1 μm to 5 μm. The membrane may have a thickness of 500 μm or greater, 250 μm or greater, 100 μm or greater, 80 μm or greater, 50 μm or greater, or 30 μm or greater. The membrane may have a thickness of 2500 μm or less, 1000 μm or less, 500 μm or less, 250 μm or less, or 100 μm or less. The thickness of the membrane may be in a range of 30 μm to 500 μm, 50 μm to 500 μm, 80 μm to 500 μm, 100 μm to 500 μm, 250 μm to 500 μm, 30 μm to 250 μm, 50 μm to 250 μm, 80 μm to 250 μm, 100 μm to 2500 μm, 30 μm to 100 μm, 50 μm to 100 μm, or 80 μm to 100 μm.

The membranes may be stacked into a multi-layer arrangement to increase capacity for a given application. In one embodiment, the stacked arrangement of membranes has a thickness of 70 μm or greater, 250 μm or greater, or 500 μm or greater. The stacked arrangement of membranes may have a thickness of 10,000 μm or less, 7,500 μm or less, 5,000 μm or less, 4,000 μm or less, 3,000 μm or less, 2,500 μm or less, 2,000 μm or less, 1,000 μm or less, 750 μm or less, 500 μm or less, 400 μm or less, or 300 μm or less. The stacked arrangement of membranes may have a thickness ranging from 70 μm to 10,000 μm, 70 μm to 100 μm, 70 μm to 200 μm, 70 μm to 300 μm, 70 μm to 400 μm, 70 μm to 500 μm, 70 μm to 750 μm, 70 μm to 1,000 μm, 70 μm to 2,000 μm, 70 μm to 3,000 μm, 70 μm to 4,000 μm, 70 μm to 5,000 μm, 250 μm to 300 μm, 250 μm to 400 μm, 250 μm to 500 μm, 250 μm to 750 μm, 250 μm to 1,000 μm, 250 to 2,000 μm, 250 to 3,000 μm, 250 to 4,000 μm, 250 to 5,000 μm, 500 μm to 1,000 μm, 500 μm to 2,000 μm, 500 μm to 3,000 μm, 500 μm to 4,000 μm, or 500 μm to 5,000 μm in thickness.

In one preferred embodiment, the membrane is a regenerated cellulose membrane having a pore size of between 0.2 μm and 5.0 μm, a thickness of between 70 μm and 2,000 μm, in a stacked arrangement approximately 70 μm to 10,000 μm in thickness.

The substrate may be a microfiltration membrane. Microfiltration membranes are typically created through a phase inversion process or an expansion process. Typical materials used to prepare membranes include PES, Nylon, PVDF, cellulose acetate, regenerated cellulose, polypropylene, and expanded PTFE.

In some cases, membranes cannot tolerate a wide range of organic solvents. The membrane and ligands may be selected so that the membrane is not soluble in the solvent used in the separation or purification process.

In embodiments where the target molecule is a nucleotide or modified nucleotide, solvents, salts, or other additives may be added to the solution containing the target molecule during the purification process to screen any repulsion of the associated phosphate groups sufficiently to allow for hybridization to occur. A different solvent or a low conductivity buffer may be implemented to elute the target molecule from the immobilized base via charge repulsion between the target and the ligands. Suitable solvents include, for example, methanol, ethanol, isopropanol, and acetonitrile, DMSO, and DMF, and the like. In some embodiments, ethanol, isopropanol, or acetonitrile is added to the solution. Suitable solvents may be used during the purification process in an amount of 5 wt-% or greater, 10 wt-% or greater, 20 wt-% or greater, 30 wt-% greater, 40 wt-% or greater, 50 wt-% or greater, 60 wt-% or greater, 70 wt-% or greater, 80 wt-% or greater or 90 wt-% or greater by weight of the solution. Suitable solvents may be used in an amount of 90 wt-% or less, 80 wt-% or less, 70 wt-% or less, 60 wt-% or less, 50 wt-% or less, 40 wt-% or less, 30 wt-% or less or 20 wt-% or less by weight of the solution. The solvents may be used in an amount ranging from 10 wt-% to 90 wt-%, 20 wt-% to 80 wt-%, 30 wt-% to 70 wt-% or 10 wt-% to 50 wt-% by weight of the solution.

Suitable salts that may be included in the solution include, for example, sodium chloride, potassium chloride, lithium chloride, rubidium chloride, calcium chloride, magnesium chloride, cesium chloride, tris base, sodium phosphate, potassium phosphate, and ammonium sulfate, etc. In some embodiments, sodium chloride, potassium chloride, ammonium sulfate, calcium chloride, potassium chloride, or magnesium chloride is added to the solution. Suitable salts may be added in an amount of 2 wt-% or greater, 5 wt-% or greater, 10 wt-% or greater, 15 wt-% or greater, or 20 wt-% or greater by weight of the solution. Suitable salts may be added in an amount of 20 wt-% or less, 25 wt-% or less, or 30 wt-% or less by weight of the solution. The salts may be added in an amount ranging from 2 wt-% to 30 wt-% or 5 wt-% to 25 wt-%, or 5 wt-% to 20 wt-% by weight of the solution.

In some embodiments where the target molecule is a nucleoside/nucleobase or modified nucleoside/nucleobase (as opposed to a nucleotide or modified nucleotide), electrostatic repulsion becomes a lesser factor, as the nucleoside does not contain a phosphate group, and therefore techniques that mitigate phosphate-phosphate repulsion become less relevant.

If the target molecules includes modifications to the groups involved in hydrogen bonding or in a sterically hindering location, it is possible to separate out unmodified and modified groups using the functionalized substrate (e.g., functionalized membrane), as modified groups will behave differently in binding in a way that can be exploited to either capture the modified molecules and let unmodified molecules flow through, or capture the unmodified molecules and let the modified molecules flow through. Hydrogen bonding competing additives or solvents may be used during elution. Examples of hydrogen bonding competing additives and solvents include acetonitrile, alcohols, water, sugar, and combinations thereof.

In many embodiments, the target molecules are present in an aqueous solution. However, in some embodiments, hydrogen bonding between A/T, A/U, and C/G remains effective even in non-aqueous environments for a variety of solvents. Examples of such solvents include alcohols, acetonitrile, and combinations thereof. Therefore, in some embodiments, the target molecules are present in a solution that includes organic solvents, such as one or more alcohols or acetonitrile. In some such embodiments the solution includes water and organic solvents. A majority of the solution maybe water. Alternatively, a majority of the solution may be made up of organic solvents. In some embodiments, the solution is nonaqueous, e.g., consists of organic solvents.

In some embodiments, the target molecule includes modifications that reduce the aqueous solubility of the target molecule (e.g., nucleoside or nucleotide). In such embodiments, an aqueous buffer-organic solvent mixture may be employed to assist in keeping the target molecule in solution (especially for hydrophobic modifications) to enhance process productivity.

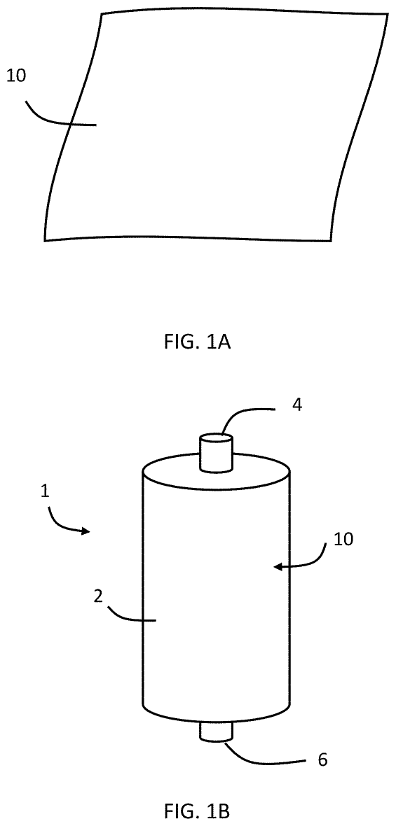

According to an embodiment, the separation media (functionalized substrate) may be used to purify target molecules at fast flow rates. For example, the separation media may be used to purify target molecules at residence times of 2 minutes or less, 1 minute (60 s) or less, 30 seconds or less, 10 seconds or less, or 6 seconds or less. Although there is no desired lower limit for the residence time, in practice residence times are 1 second or greater. The separation media may be arranged as a membrane chromatography column, a membrane chromatography cassette, or other membrane chromatography device. A sheet of separation media 10 is schematically shown in FIG. 1A. The sheet of separation media 10 may be provided in a separation device 1 (e.g., a chromatography column), shown in FIG. 1B. The separation device 1 includes a housing 2 with an inlet 4 and an outlet 6 to facilitate flow through the device. The separation device (e.g., membrane chromatography column, membrane chromatography cassette, or other membrane chromatography device) may provide a residence time of 2 minutes or less, 1 minute or less, 30 seconds or less, 10 seconds or less, or 6 seconds or less. According to an embodiment, using membrane-based purification devices can significantly improve productivity.

Process productivity can be defined using the equation below. In the denominator, Vₜₒₜ is the total volume of solution passing through the separation media (e.g., column or cassette) during the whole process, including load, rinse, elution, and regeneration steps. BV is the chromatography medium bed volume (corresponding to the volume of the separation media substrate), and τ is residence time. Loading volume is proportional to dynamic binding capacity of the chromatography column medium. Thus, process productivity increases with increasing binding capacity and decreasing residence time.

$$Productivity = \frac{drug captured}{Cost of time} = \frac{Loading volume ⨯ drug concentration ⨯ yield}{( \frac{{V}_{tot}}{BV} ) ⨯ τ}$$

According to another embodiment, the separation media includes a cation-exchange substrate. Such a substrate may be used in cation-exchange based chromatography to purify the target molecules (e.g., nucleosides, nucleotides, nucleobases, or their analogues or derivatives). Cation-exchange employs negatively charged functional groups that target positively charged target molecules.

The cation-exchange ligand may be conjugated to the substrate via a functional handle. The functional handle may covalently bond with the substrate. In some embodiments, cation-exchange ligands are prepared from difunctional molecules. One of the functional groups may act as the functional handle. In general, the ligands, including cation-exchange ligands, may have the following general formula (I)

Fh-Sp-Sg (I)

where Fh is the functional handle, Sp is a spacer, and Sg is a functional separation group, e.g., a cation-exchange separation group. The functional handle allows the ligand (e.g., cation exchange ligand) to be conjugated to the substrate.

The cation-exchange separation group is a functional group that allows for the separation of nucleic acids, nucleotides, one of more components of nucleotides, analogues thereof, or derivatives thereof. In embodiments, the cation-exchange separation group may include one cation-exchange separation moiety or two cation-exchange separation moieties.

The spacer separates the functional handle from the cation-exchange separation group. The spacer may be of a length and/or composition that allows for the functional handle and/or the cation-exchange separation group to function as intended.

The functional handle may include any reactive functional group that may undergo a reaction with a chemical moiety that is on the substrate to form a covalent bond. Reaction of the functional handle with a substrate reactive moiety, a reactive moiety on the substrate, results in the covalent attachment of the cation-exchange ligand to the substrate, also termed a conjugated cation-exchange ligand. The conjugated cation-exchange ligand may be displayed on the substrate to allow for separation of nucleic acids, nucleotides, one of more components of nucleotides, analogues thereof, or derivatives thereof.

The identity of the reactive functional group of the functional handle is informed from the identity of the substrate reactive moiety, that is, the reactive functional handle and the substrate reactive moiety must be compatible to undergo a reaction to form a covalent bond. Example substrate reactive moieties and/or example reactive functional groups include amines; alcohols; activated alcohols such as tosyl protected alcohols (e.g., tosyl chloride); epoxides; isocyanates; alkenes; alkynes; cycloalkenes; cyclooctynes; thiols; disulfides; azides; thioisocyanate; N-hydroxysuccinimide; maleimides; and activated esters and/or carboxylic acids including esters or carboxylic acids activated using carbodiimide compounds (N,N′-dicyclohexylcarbodiimide, N,N′-diisopropylcarbodiimide, 1-ethyl-3-(3-dimethylaminopropyl)carbodiimide, and 1-cyclohexyl-(2-morpholinoethyl)carbodiimide, metho-p-toluene sulfonate. A person of skill would understand which reactive functional handles and substrate reactive moieties would be compatible.

Upon conjugation to the substrate, the conjugated cation-exchange ligand may have the general formula (II):

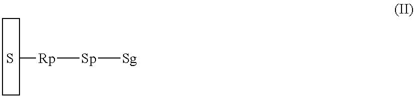

where S is the substrate, Rp is the reaction product between the functional handle and the substrate reactive moiety, Sp is the spacer, and Sg is the separation group. In some embodiments, the Rp may be a second separation group that facilitates separation of nucleic acids, nucleotides, one of more components of nucleotides, analogues thereof, or derivatives thereof. In some embodiments where Rp is not a separation group and the first separation group (e.g., Sg) includes a single separation moiety, the conjugated ligand is a monomodal ligand, such as a cation-exchange monomodal ligand. The separation group may be an acid, a carboxylic acid, a sulfonic acid, a phosphoric acid, a carboxylate, a sulfonate, or a phosphate. In some embodiments where Rp is not a separation group and the first separation group (e.g., Sg) includes two separation moieties, the conjugated ligand is a bimodal ligand, such as a conjugated cation-exchange bimodal ligand. In some embodiments where Rp is a second separation group and the first separation group (e.g., Sg) includes a single separation moiety, the conjugated ligand is a bimodal ligand, such as a conjugated cation-exchange bimodal ligand. In some embodiments where Rp is a second separation group and the first separation group (e.g., Sg) includes two separation moieties (e.g., a first separation moiety and a second separation moiety), the conjugated ligand is a trimodal ligand, such as a conjugated cation-exchange trimodal ligand.

The identity of the reaction product depends on the identity of the reactive functional group and the substrate reactive moiety. Example, reaction products include, but are not limited to esters, ethers, thioethers, amides, amines (e.g., primary, secondary, tertiary), alkenes, urea, carbamate, carbonate, thiourea, and triazoles.

In some embodiments, the separation group (Sg) may include a single separation moiety.

In some embodiments, the separation group (Sg) may include two separation moieties and be of the general formula (III)

Sm1-Sp₂-Sm2 (III)

where Sm1 is the first separation moiety, Sp2 is a spacer, and Sm2 is a second separation moiety. The first and second separation moieties may be an acid, a carboxylic acid, a sulfonic acid, a phosphoric acid, a carboxylate, a sulfonate, or a phosphate. The spacer (Sp/Sp2) may be a carbon chain of length C1 to C18, C1 to C10, C1 to C6, C1 to C4, C1 to C3, or C2 to C4, optionally substituted with one or more ethers, esters, benzyls, phenyls, or amides along the carbon chain. Example separation groups that include two separation moieties include aminobenzoic acid, aminodiacetic acid, aminopropanoic acid, 3-amino-1-propanesulfonic acid, and 3-amino-1-ethylsulfonic acid. The cation-exchange substrate may be prepared by first subjecting (e.g., immersing) a base substrate (e.g., a membrane or a nonwoven substrate) to a solution of linker activation agent and catalyst, then subjecting (e.g., immersing) the substrate to a solution containing the ligand and optionally a catalyst, and finally immersing the substrate in a quenching buffer solution to passivate unreacted linkers. In one exemplary embodiment, the linker activation agent is or includes N,N-disuccimidylcarbonate (DSC) and the catalyst includes triethylamine (TEA).

In embodiments where the target molecule is a nucleobase/nucleoside or modified nucleobase/nucleoside, suitable solvents, salts, or other additives may be added to the solution containing the target molecule to allow the target molecule to be dissolved or maintain its stability, or achieve desired level of binding and selectivity.

Suitable salts used during the purification process include, for example, sodium chloride, potassium chloride, lithium chloride, rubidium chloride, calcium chloride, magnesium chloride, cesium chloride, tris base, sodium phosphate, potassium phosphate, and ammonium sulfate, etc. In some embodiments, sodium chloride, potassium chloride, ammonium sulfate, calcium chloride, potassium chloride, or magnesium chloride is added to the solution. Suitable salts may be added in an amount of 1 mM or greater, 5 mM or greater, or 10 mM or greater, or 20 mM or greater. Suitable salts may be added in an amount of 100 mM or less, 50 mM or less, or 30 mM or less. The salts may be added in an amount ranging from 1 mM to 100 mM, 1 mM to 50 mM, 5 mM to 30 mM, or 5 mM to 20 mM.

Suitable solvents used during the purification process include, for example, methanol, ethanol, isopropanol, and acetonitrile, DMSO, and DMF, etc. In some embodiments, ethanol, isopropanol, or acetonitrile is added to the solution. Suitable solvents may be added in an amount of 5 wt-% or greater, 10 wt-% or greater, 20 wt-% or greater, 30 wt-% greater, 40 wt-% or greater, 50 wt-% or greater, 60 wt-% or greater, 70 wt-% or greater, 80 wt-% or greater or 90 wt-% or greater. Suitable solvents may be added in an amount of 90 wt-% or less, 80 wt-% or less, 70 wt-% or less, 60 wt-% or less, 50 wt-% or less, 40 wt-% or less, 30 wt-% or less or 20 wt-% or less. The solvents may be added in an amount ranging from 10 wt-% to 90 wt-%, 20 wt-% to 80 wt-%, 30 wt-% to 70 wt-% or 10 wt-% to 50 wt-%.

A different solvent or a higher conductivity buffer may be implemented to elute the target molecule from the immobilized base via charge repulsion between the target and the ligands.

Cytosine (C) and guanine (G) bases contain a protonatable primary amine at position 4 of the pyrimidine ring. The charge state of this amine can be protonated to a positive charge, which can then provide selectivity using a cation-exchange chromatography (CEX) substrate (e.g., membrane). Other nucleobase charge states may also be manipulated by manipulating the pH of the solution. For example, the pKa of the amine at position at position 4 of cytosine is about 4.45. For guanine the pKa of the amine at position 2 is about 12.3, for the amine at position 9 is about 9.2, and amide at position 1 is about 3.3. The pKa of the amide in thymidine is about 9.96. The pKa of the amine in adenosine is about 3.5. The pH of the solution may be adjusted to be below the pKa of the nucleobase to protonate the amine and to take advantage of cation-exchange chromatography.

When target molecules are separated or purified using cation-exchange chromatography, the pH of the solution may be monitored and controlled such that it stays below the pKa of the target molecule to maintain the molecule in a protonated state. The pH may also be maintained above a threshold to avoid target decomposition. The pH threshold may vary from molecule to molecule. For example, BOC was used to protect reactive groups, which can be deprotected at a very acidic condition, such as 4 M HCl in dioxane or 1 M HCl in acetic acid.

In some embodiments, alcohol and/or hydroxyl groups on target molecules may be protected from synthetic attack to allow for targeted conjugation of amines. The protecting groups may typically be removed using an acid solution. During purification, it may be desirable to monitor and control the acidity of the solution to maintain the pH above the pH of deprotection (often performed with trichloroacetic acid or hydrochloric acid). On the other hand, to induce or maintain protonation of the target molecule in order to utilize cation-exchange separation, pH of the solution may be maintained below the pKa of the target molecule.

Molecules coupled with cation-exchange ligands may be eluted, for example, by raising the conductivity, screening the electrostatic attraction between the CEX chromatography media and the target molecules. In one embodiment, elution can be performed by altering the pH above the pKa of the amine in the target molecule, forming a neutral charge in the target molecule, thus reducing the charge interaction and inducing elution. Different target molecules (or target molecules and other molecules) may be eluted using a linear gradient elution or using a step isocratic elution.

The substrate used to prepare the cation-exchange chromatography (CEX) substrate may be any suitable material. In some embodiments, the functionalized substrate is or includes a membrane, resin, monolith, hydrogel, woven fibrous substrate, nonwoven fibrous substrate, or a combination thereof. In one embodiment, the functionalized substrate is or includes a membrane. In one embodiment, the functionalized substrate is or includes a woven or nonwoven fibrous substrate. Suitable membranes and nonwoven fibrous substrates are discussed elsewhere in this disclosure.

According to an embodiment, the cation-exchange substrate may be used to purify target molecules at fast flow rates. For example, the cation-exchange substrate may be used to purify target molecules at residence times of 2 minutes or less, 1 minute or less, 30 seconds or less, 10 seconds or less, or 6 seconds or less. Although there is no desired lower limit for the residence time, in practice residence times are 1 second or greater. The cation-exchange substrate may be arranged as a membrane chromatography column, a membrane chromatography cassette, or other membrane chromatography device. The membrane chromatography column, membrane chromatography cassette, or other membrane chromatography device may provide a residence time of 2 minutes or less, 1 minute or less, 30 seconds or less, 10 seconds or less, or 6 seconds or less. According to an embodiment, using membrane-based purification devices can significantly improve productivity.

In some embodiments, the separation media includes an anion-exchange substrate. Such a substrate may be used in anion-exchange based chromatography to purify the target molecules (e.g., nucleosides, nucleotides, nucleobases, or their analogues or derivatives). Anion exchange employs positively charged functional groups that target negatively charged target molecules.

The anion-exchange ligand may be conjugated to the substrate via a functional handle. The functional handle may covalently bond with the substrate. In some embodiments, anion-exchange ligands are prepared from difunctional, trifunctional, or other multifunctional molecules. One of the functional groups may act as the functional handle. The functional handle may be as described above with regard to formula (I). The conjugated anion-exchange ligand may include a reaction product Rp and spacer Sp as described above with regard to formula (II). The conjugated anion-exchange ligand further includes a separation group Sg.

Examples of suitable anion exchange ligands that may be disposed on the separation media substrate include primary, secondary, tertiary, and quaternary amines. Suitable amines may be diamines, triamines, and polyamines. Diamines are generally represented by the following formula:

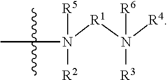

In some embodiments, R¹ is an aliphatic carbon chain of 1 to 18 carbons, 1 to 10 carbons, 1 to 6 carbons, or 2 to 4 carbons. In quaternary amines, each one of R², R³, R⁴, R⁵, and R⁶ are individually selected from aliphatic straight chain, branched chain, or cyclic, substituted or non-substituted, carbon chains having a length of 1 to 10 carbons, 1 to 6 carbons, or 2 to 4 carbons. In tertiary amines, R⁵ and R⁶ are absent, and R², R³, and R⁴ are as in quaternary amines. In secondary amines, R⁵ and R⁶ are absent, R² and R³ are H, and R⁴ is as in quaternary amines. In primary amines, R⁵ and R⁶ are absent and R², R³, and R⁴ are H. In some embodiments, the nitrogens of the diamine have different levels of substitution. For example, one amine may be a secondary amine and one amine may be primary, tertiary, or quaternary. Suitable triamines and polyamines may have an analogous structure with three (triamine) or more amine groups.

Examples of primary amines include methylene diamine, ethylene diamine, propylene diamine, butylenediamine (putrescine), pentylamine, or any aliphatic diamine with 1-18 carbons between the terminal amines, covalently attached via one of the amines. Such ligands can be made from polyamines such as ethylene diamine, diethylenetriamine, triethylenetetramine covalently attached via one of the amines.

Examples of secondary amines can include any of the aforementioned primary amines immobilized to the substrate, substituted with an additional R-group as described above. In cases in which diamines are used, secondary amines may also be formed by covalent interaction with the substrate coupling both amines to the substrate. Ligands containing secondary amines with the structure of the ligand may also be immobilized such as linear polyethyleneimine, spermidine, or spermine. Furthermore, groups containing a non-terminal primary amine (e.g., 3-aminopentane) may also be conjugated to the substrate to result in a secondary amine.

Examples of suitable tertiary amines include N,N-dimethylethylenediamine, N,N-dimethylpropylenediamine N,N-diethylpropylenediamine or any aliphatic diamine with aliphatic carbon group substitution on one or both amines ranging from one to six carbons, with an R¹ having 2-18 carbons between the terminal amines.

Examples of quaternary amines include any of the aforementioned primary amines that have undergone a quaternarization reaction resulting in a permanent positive charge. Such reactions can be performed with alkyl groups such as methyl iodide or aryl groups such as benzyl iodide. Quaternary amines can further include any of the aforementioned tertiary amines that have undergone a quaternarization reaction resulting in a permanent positive charge. Such reactions can be described by the Menshutkin reaction which uses an alkyl halide to form a quaternary ammonium salt from a reaction with a tertiary amine. Such reactions can be performed with alkyl containing groups of varying length such as butyl bromide or aryl groups such as benzyl chloride or combinations therein. Additionally, compounds containing quaternary amines can be immobilized directly.

The ligand may include additional functional groups in addition to amine groups. The ligand may include a linker between the amine and any other functionalities that is 1 to 18 carbons, 1 to 10 carbons. 1 to 6 carbons, or 2 to 4 carbons long.

The anion-exchange substrate may be prepared by first subjecting (e.g., immersing) a base substrate (e.g., a membrane or a nonwoven substrate) to a solution of linker activation agent and catalyst, then subjecting (e.g., immersing) the substrate to a solution containing the ligand and optionally a catalyst, and finally subjecting (e.g., immersing) the substrate to a buffer solution. In one exemplary embodiment, the linker activation agent is or includes N,N-disuccimidylcarbonate (DSC) and the catalyst includes triethylamine (TEA).

In one exemplary embodiment, the anion-exchange substrate is made in a three-step process. The first step includes subjecting (e.g., immersing) a base substrate to a solution of linker activation agent and catalyst. This may include from 0.1 mg/mL to 120 mg/mL of DSC, and 5 μL/mL to 100 μL/mL of TEA in solvent. The solvent may include DMSO, acetonitrile, tetrahydrofuran (THF), dimethylformamide (DMF), hexamethylphosphoramide, sulfolane, or any other solvent/solution that swells the substrate (e.g., membrane). The subjecting may be done at a temperature of between about 10° C. to 60° C. for about 1 minute to 1,800 minutes. For example, a membrane having a diameter of 47 mm and a thickness of 70 μm may be soaked in 300 mg of DSC, 139 μL of TEA, dissolved in 10 mL of DMSO at 40° C. for 16 hours.

In this exemplary embodiment, the second step includes subjecting (e.g., immersing) the substrate to a solution containing the ligand and optionally a catalyst. This may include from about 1 to 100 μL/mL, <100 μL/mL, <75 μL/mL, <50 μL/mL, <20 μL/mL, <10 μL/mL, 1 μL/mL, to 10 μL/mL, 1 μL/mL, to 20 μL/mL, 1 μL/mL to 50 μL/mL, 1 μL/mL to 75 μL/mL, 1 μL/mL, to 100 μL/mL, 10 μL/mL, to 20 μL/mL, 10 μL/mL, to 50 μL/mL, 10 to 75 μL/mL to 100 μL/mL, 20 μL/mL to 50 μL/mL, 20 μL/mL, to 75 μL/mL, 20 μL/mL to 100 μL/mL, 50 μL/mL, to 75 μL/mL, or 50 μL/mL to 100 μL/mL, of DMEDA in solvent. The solvent may be DMSO or another organic solvent such as acetonitrile, THF, hexamethylphosphoramide, sulfolane, or any other solvent/solution that swells the substrate. The subjecting may be done at a temperature of between about 10° C. to 60° C. for about 1 minute to 24 hours. For example, the membrane may be placed in a solution of 15 μL/mL of DMEDA in DMSO at room temperature for 30 minutes.

In this exemplary embodiment, the third includes subjecting (e.g., immersing) the substrate from step 2 to a buffer solution. This may include 0.05 M to 4 M Tris at pH 7.0-10.0. The subjecting may be done at a temperature of between about 10° C. to 60° C. for about 1 minute to 24 hours. For example, the membrane is placed in 1 M Tris pH 8.0 for 16 hours.

In some embodiments, the anion-exchange substrate is prepared according to Method 1 described in US20200188859A1 (Zhou et al.).

In embodiments where the target molecule is a nucleotide or modified nucleotide, solvents, salts, or other additives may be added to the solution containing the target molecule to allow the target molecule to be dissolved or maintain its stability, or achieve a desired level of binding and selectivity.

Suitable salts used during the purification process include, for example, sodium chloride, potassium chloride, lithium chloride, rubidium chloride, calcium chloride, magnesium chloride, cesium chloride, tris base, sodium phosphate, potassium phosphate, and ammonium sulfate, etc. In some embodiments, sodium chloride, potassium chloride, ammonium sulfate, calcium chloride, potassium chloride, or magnesium chloride is added to the solution. Suitable salts may be added in an amount of 1 mM or greater, 5 mM or greater, or 10 mM or greater, or 20 mM or greater. Suitable salts may be added in an amount of 100 mM or less, 50 mM or less, or 30 mM or less. The salts may be added in an amount ranging from 1 mM to 100 mM, 1 mM to 50 mM, 5 mM to 30 mM, or 5 mM to 20 mM.

Suitable solvents used during the purification process include, for example, methanol, ethanol, isopropanol, and acetonitrile, DMSO, and DMF, etc. In some embodiments, ethanol, isopropanol, or acetonitrile is added to the solution. Suitable solvents may be added in an amount of 5 wt-% or greater, 10 wt-% or greater, 20 wt-% or greater, 30 wt-% greater, 40 wt-% or greater, 50 wt-% or greater, 60 wt-% or greater, 70 wt-% or greater, 80 wt-% or greater or 90 wt-% or greater. Suitable solvents may be added in an amount of 90 wt-% or less, 80 wt-% or less, 70 wt-% or less, 60 wt-% or less, 50 wt-% or less, 40 wt-% or less, 30 wt-% or less or 20 wt-% or less. The solvents may be added in an amount ranging from 10 wt-% to 90 wt-%, 20 wt-% to 80 wt-%, 30 wt-% to 70 wt-%, or 10 wt-% to 50 wt-%.

A different solvent or a higher conductivity buffer may be implemented to elute the target molecule from the immobilized base via charge repulsion between the target and the ligands.

According to an embodiment, the pH of the feed solution containing the target is adjusted to maintain the target molecule positively charged, which pH is usually higher than the pKa of such molecule. On the other hand, the pH of the feed solution is maintained lower than the pKa of the anion-exchange membrane ligands to maintain its positive status. For example, if a weak anion-exchange membrane is used to capture adenosine monophosphate from a feed solution, the pH of the feed solution may be adjusted to be in a range from 3 to 7.

In some embodiments, the separation media includes a substrate with functional groups that induce hydrophobic interactions with the target molecules, impurities, or both. Such a substrate may be used in hydrophobic interaction chromatography (HIC) to purify the target molecules (e.g., nucleosides, nucleotides, nucleobases, or their analogues or derivatives). Hydrophobic interaction chromatography employs hydrophobic functional groups that interact with hydrophobic groups on the target molecules. Hydrophobic interactions exploit the differences in hydrophobicity of between the target molecules and possible impurities. Nucleobases contain hydrophobic rings that can be exploited by interacting with the HIC ligands on the substrate.

In one embodiment, such ligands include aliphatic chains with three carbons or longer (common used lengths include butyl, pentyl, hexyl, heptyl, octyl, nonyl, decyl, undecyl, and dodecyl), benzyl, phenyl, phenol, pyridine, boronic acid groups, branched polymers such as polypropylene glycol, and sulfur-containing thiophilic ligands such as propanethiol, 2-butanethiol, 3,6-dioxa-1,8-octanedithiol, octanethiol, benzyl mercaptan, 2-mercaptopyridine, thiophenol, 1,2-ethanedithiol, 1,4-benzenedimethanethiol, 2-phenylethanethiol, and the like, and combinations thereof. The hydrophobic interaction ligand may be conjugated with the substrate via a functional handle as described above with regard to formula (I).

In some embodiments the target molecule is a nucleobase or modified nucleobase, nucleoside or modified nucleoside, or nucleotide or modified nucleotide. Solvents, salts, or other additives may be added to the solution containing the target molecule to allow for binding to occur through interaction of the hydrophobic groups present on both the ligand and the target. In some embodiments, kosmotropic salts are added to the solution. In some cases, a combination of kosmotropic and chaotropic salts may be added to the solution. For example, a mixture of kosmotropic anions and chaotropic cations may be used. In some embodiments, the proportion of kosmotropic salts is increased and/or the proportion of chaotropic salts is decreased. Kosmotropic salts are known as salts that decrease the solubility of nonpolar substances in aqueous solutions, while chaotropic salts increase their solubility. In some embodiments, the proportion of organic solvent in the solution may be increased. In other embodiments, the proportion of organic solvent in the solution may be decreased. In other embodiments, a combination of alterations of kosmotropic, components, chaotropic components, and/or organic solvents may also be used.

Examples of kosmotropic salts that may be added to the solution containing the target molecules include ammonium sulfate, ammonium phosphate, potassium phosphate, sodium sulfate, sodium chloride, and combinations thereof. Suitable kosmotropic salts may be added in an amount of 0.1 M or greater, 0.5 M or greater, or 1.0 M or greater, or 2.0 M or greater. Suitable kosmotropic salts may be added in an amount of 6.0 M or less, 5.0 M or less, or 4.0 M or less. The kosmotropic salts may be added in an amount ranging from 0.1 M to 6M, 0.5 M to 2.5 M, or 0.5 M to 3.0 M.

Examples of chaotropic salts that may be present in the solution include sodium chloride, calcium chloride, magnesium chloride and combinations thereof. In some embodiments, the amount of chaotropic salts is maintained at 1 M or less, 0.5 M or less, or 0.1 M or less. In some embodiments, the solution is free or substantially free of chaotropic salts.

Suitable solvents used during the purification process include, for example, methanol, ethanol, isopropanol, and acetonitrile, DMSO, and DMF, etc. In some embodiments, ethanol, isopropanol, or acetonitrile is added to the solution. Suitable solvents may be added in an amount of 5 wt-% or greater, 10 wt-% or greater, 20 wt-% or greater, 30 wt-% greater, 40 wt-% or greater, 50 wt-% or greater, 60 wt-% or greater, 70 wt-% or greater, 80 wt-% or greater or 90 wt-% or greater. Suitable solvents may be added in an amount of 90 wt-% or less, 80 wt-% or less, 70 wt-% or less, 60 wt-% or less, 50 wt-% or less, 40 wt-% or less, 30 wt-% or less or 20 wt-% or less. The solvents may be added in an amount ranging from 10 wt-% to 90 wt-%, 20 wt-% to 80 wt-%, 30 wt-% to 70 wt-% or 10 wt-% to 50 wt-%.

A different solvent or a low conductivity buffer may be implemented to elute the target molecule from the immobilized base via charge repulsion between the target and the ligands.

The substrate used to prepare the hydrophobic interaction chromatography (HIC) substrate may be any suitable material. In some embodiments, the hydrophobic interaction chromatography (HIC) substrate is or includes a membrane, resin, monolith, hydrogel, and fibers, etc. In one embodiment, the hydrophobic interaction chromatography (HIC) substrate is or includes a membrane. In one embodiment, the hydrophobic interaction chromatography (HIC) substrate is or includes a nonwoven fibrous substrate.

According to an embodiment, the hydrophobic interaction substrate may be used to purify target molecules at fast flow rates. For example, the hydrophobic interaction substrate may be used to purify target molecules at residence times of 2 minutes or less, 1 minute or less, 30 seconds or less, 10 seconds or less, or 6 seconds or less. Although there is no desired lower limit for the residence time, in practice residence times are 1 second or greater. The hydrophobic interaction substrate may be arranged as a membrane chromatography column, a membrane chromatography cassette, or other membrane chromatography device. The membrane chromatography column, membrane chromatography cassette, or other membrane chromatography device may provide a residence time of 2 minutes or less, 1 minute or less, 30 seconds or less, 10 seconds or less, or 6 seconds or less. According to an embodiment, using membrane-based purification devices can significantly improve productivity.

In some embodiments, the separation media includes multimodal media. Multimodal media is media that includes two or more types of ligands or functional groups on the substrate. Multimodal media may enhance the interaction between the ligand and target molecule. In some embodiments, the multimodal media includes an ion exchange ligand or functional group and one other type of ligand or functional group. In some such embodiments, the multimodal media includes cation exchange ligands and at least one other type of functional group. In some embodiments, the multimodal media includes anion exchange ligands and at least one other type of functional group. The at least one other type of functional group may be part of the same ligand as the cation or anion exchange group, or may be in a separate ligand. For example, the multimodal media may further include hydrophobic interaction groups, hydrogen bonding groups, thiophilic groups, or a combination thereof, in addition to cation exchange ligands or anion exchange ligands. In one embodiment, the multimodal media includes a combination of cation exchange ligands and hydrophobic interaction groups. In one embodiment, the multimodal media includes a combination of cation exchange ligands and hydrogen bonding groups. In one embodiment, the multimodal media includes a combination of cation exchange ligands and thiophilic groups. The multimodal media may also include three or more types of functional groups. Multimodal medias may be used to separate or purify nucleotides, nucleosides, nucleobases, and their analogues and derivatives.

According to an exemplary embodiment, a multimodal media includes ligands containing a cation-exchange ligand and a hydrophilic ligand.

According to an exemplary embodiment, a multimodal media includes ligands containing a cation-exchange ligand and a hydrophobic interaction ligand.

According to an exemplary embodiment, a multimodal media includes ligands containing an anion-exchange ligand and a hydrophilic ligand.

According to an exemplary embodiment, a multimodal media includes ligands containing an anion-exchange ligand and a hydrophobic interaction ligand.

In some examples, the multimodal media includes cation-exchange ligands that also include thiophilic functionality. Examples of suitable thiophilic cation-exchange ligands that may be disposed on the separation media substrate include mercaptocarboxylic acids and their salts. Thiophilic cation-exchange ligands may be represented by the following formula:

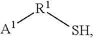

where R₁ is a spacer group. R¹ may be an aliphatic or aromatic group, substituted or un-substituted, containing 1 to 18 carbons, 1 to 10 carbons, 1 to 6 carbons, or 2 to 4 carbons. R¹ may be straight chain, branched, or cyclic. A¹ is a carboxylic acid (optionally conjugated to an aromatic group, such as at the benzoic or benzylic position) or a sulfonate group.

Examples of suitable thiophilic cation-exchange ligands include mercaptobenzoic acid (e.g., 2-mercaptobenzoic acid or 4-mercaptobenzoic acid), and mercaptosulfonic acids and their salts, such as sodium 3-mercapto-1-propanesulfonate. The ligand may include a linker between the sulfur-containing and other (e.g., acid) functionalities that is 1 to 18 carbons, 1 to 10 carbons, 1 to 6 carbons, or 2 to 4 carbons long.

The thiophilic cation-exchange substrate may be prepared by first subjecting (e.g., immersing) a base substrate (e.g., a membrane or a nonwoven substrate) to a solution of linker activation agent and catalyst, then subjecting (e.g., immersing) the substrate to a solution containing the ligand and optionally a catalyst, and finally immersing the substrate in a buffer solution. In one exemplary embodiment, the linker activation agent is or includes N,N-disuccimidylcarbonate (DSC) and the catalyst includes triethylamine (TEA).

In one exemplary embodiment, the thiophilic cation-exchange substrate is made in a three-step process. The first step includes subjecting (e.g., immersing) a base substrate to a solution of linker activation agent and catalyst. This may include from 0.1 mg/mL to 120 mg/mL of DSC, and 5 μL/mL, to 100 μL/mL, of TEA in solvent. The solvent may include DMSO, acetonitrile, tetrahydrofuran (THF), dimethylformamide (DMF), hexamethylphosphoramide, sulfolane, or any other solvent/solution that swells the substrate (e.g., membrane). The subjecting may be done at a temperature of between about 10° C. to 60° C., for about 1 minute to 1,800 minutes. For example, a membrane having a diameter of 47 mm and a thickness of 70 μm may be soaked in 300 mg of DSC, 139 μL of TEA, dissolved in 10 mL of 10 mL of DMSO at 40° C. for 16 hours.

In this exemplary embodiment, the second step includes subjecting (e.g., immersing) the substrate to a solution containing the ligand and optionally a catalyst. This may include from about 0.1 mg/mL to 150 mg/mL, from 1 mg/mL to 100 mg/mL, or from 10 mg/mL to 50 mg/mL of sodium 3-mercapto-1-propanesulfonate in solvent. The solvent may be DMSO or another organic solvent such as acetonitrile, THF, DMF, hexamethylphosphoramide, sulfolane, or any other solvent/solution that swells the substrate. The subjecting may be done at a temperature of between about 10° C. to 60° C. for about 1 minute to 24 hours. For example, the membrane may be placed in a solution of 300 mg of sodium 3-mercapto-1-propanesulfonate, 1 mL, of TEA, dissolved in 10 mL, of DMSO) at 40° C. for 16 hours.

In this exemplary embodiment, the third step includes subjecting (e.g., immersing) the substrate from step 2 to a buffer solution. This may include 0.05 M to 4 M Tris at pH 7.0-10.0. The subjecting may be done at a temperature of between about 10° C. to 60° C. for about 1 minute to 24 hours. For example, the membrane is placed in 1 M tris(hydroxymethyl)aminomethane (Tris) pH 8.0 for 16 hours.

In some embodiments, the desired target is a nucleobase or modified nucleobase, nucleoside or modified nucleoside, or nucleotide or modified nucleotide. Suitable solvents, salts, or other additives may be added to the solution to allow the target molecule to be dissolved or maintain its stability, or achieve a desired level of binding and selectivity.

Suitable salts used during the purification process include, for example, sodium chloride, potassium chloride, lithium chloride, rubidium chloride, calcium chloride, magnesium chloride, cesium chloride, tris base, sodium phosphate, potassium phosphate, and ammonium sulfate, etc. In some embodiments, sodium chloride, potassium chloride, ammonium sulfate, calcium chloride, potassium chloride, or magnesium chloride is added to the solution. Suitable salts may be added in an amount of 1 mM or greater, 5 mM or greater, or 10 mM or greater, or 20 mM or greater. Suitable salts may be added in an amount of 100 mM or less, 50 mM or less, or 30 mM or less. The salts may be added in an amount ranging from 1 mM to 100 mM, 1 mM to 50 mM, 5 mM to 30 mM, or 5 mM to 20 mM.

Suitable solvents used during the purification process include, for example, methanol, ethanol, isopropanol, and acetonitrile, DMSO, and DMF, etc. In some embodiments, ethanol, isopropanol, or acetonitrile is added to the solution. Suitable solvents may be added in an amount of 5 wt-% or greater, 10 wt-% or greater, 20 wt-% or greater, 30 wt-% greater, 40 wt-% or greater, 50 wt-% or greater, 60 wt-% or greater, 70 wt-% or greater, 80 wt-% or greater or 90 wt-% or greater. Suitable solvents may be added in an amount of 90 wt-% or less, 80 wt-% or less, 70 wt-% or less, 60 wt-% or less, 50 wt-% or less, 40 wt-% or less, 30 wt-% or less or 20 wt-% or less. The solvents may be added in an amount ranging from 10 wt-% to 90 wt-%, 20 wt-% to 80 wt-%, 30 wt-% to 70 wt-% or 10 wt-% to 50 wt-%.

A different solvent or a higher conductivity buffer may be implemented to elute the target molecule from the immobilized base via charge repulsion between the target and the ligands.

When target molecules are separated or purified using multimodal media cation-exchange chromatography, the pH of the solution may be monitored and controlled such that it stays below the pKa of the target molecule to maintain the molecule in a protonated state. The pH may also be maintained above a threshold to prevent decomposition of the target and that is above the pKa of the multimodal ligand to maintain the ligand negatively charged. Examples of suitable pH ranges include pH 1 to 3 for cytidine and gemcitabine with or without protected alcohols.

Molecules coupled with cation-exchange ligands of a multimodal media may be eluted, for example, by raising the conductivity, screening the electrostatic attraction between the cation-exchange ligands and the target molecules. In one embodiment, elution can be performed by altering the pH above the pKa of the amine in the target molecule, forming a neutral charge in the target molecule, thus reducing the charge interaction and inducing elution. Different target molecules (or target molecules and other molecules) may be eluted using a linear gradient elution or using a step isocratic elution.

When target molecules are separated or purified using multimodal media anion-exchange chromatography, the pH of the solution may be monitored and controlled such that it stays above the pKa of the target molecule to maintain the molecule in a deprotonated state. The pH may also be maintained below a threshold that prevents the target from decomposition and also below the pKa of the multimodal ligand to maintain the ligand positively charged. Examples of suitable pH ranges include pH 3 to 10 for adenosine monophosphate purification.

Molecules coupled with anion-exchange ligands of a multimodal media may be eluted, for example, by raising the conductivity, screening the electrostatic attraction between the cation-exchange ligands and the target molecules. In one embodiment, elution can be performed by altering the pH below the pKa of the target molecule to reduce the charge interaction and inducing elution. Different target molecules (or target molecules and other molecules) may be eluted using a linear gradient elution or using a step isocratic elution.

The substrate used to prepare the multimodal media may be any suitable material. In some embodiments, the multimodal media is or includes a membrane, resin, monolith, hydrogel, and fibers, etc. In one embodiment, the multimodal media is or includes a membrane. In one embodiment, the multimodal media is or includes a nonwoven fibrous substrate.

According to an embodiment, the multimodal media may be used to purify target molecules at fast flow rates. For example, the multimodal media may be used to purify target molecules at residence times of 2 minutes or less, 1 minute or less, 30 seconds or less, 10 seconds or less, or 6 seconds or less. The residence time is somewhat dependent on the size of separation device, and in small devices, residence times may be as low as 1 second or less. Although there is no desired lower limit for the residence time, in practice residence times are 0.1 seconds or greater. The multimodal media may be arranged as a membrane chromatography column, a membrane chromatography cassette, or other membrane chromatography device. The membrane chromatography column, membrane chromatography cassette, or other membrane chromatography device may provide a residence time of 2 minutes or less, 1 minute or less, 30 seconds or less, 10 seconds or less, or 6 seconds or less. According to an embodiment, using membrane-based purification devices can significantly improve productivity.

### EXEMPLARY EMBODIMENTS

The following is a non-limiting list of exemplary embodiments according to the present disclosure.

Embodiment 1 is a separation media comprising: a membrane; and a plurality of ligands immobilized on the membrane, the plurality of ligands comprising anion-exchange ligands, cation-exchange ligands, thiophilic ligands, hydrophobic interaction ligands, hydrophilic ligands, or a combination thereof.
Embodiment 2 is the separation media of embodiment 1, wherein the separation media is configured for separation of target molecules comprising nucleotides, nucleosides, nucleobases, their derivatives and analogues, and combinations thereof, from a reaction mixture.
Embodiment 3 is the separation media of embodiment 1 or 2, wherein the separation media is configured for use with organic solvents.
Embodiment 4 is the separation media of any one of embodiments 1 to 3, wherein the plurality of ligands comprise anion-exchange ligands comprising an aliphatic diamine or triamine comprising 1 to 18 carbons between adjacent amines.
Embodiment 5 is the separation media of embodiment 4, wherein the anion exchange ligand comprises N,N-dimethylethylenediamine, N,N-dimethylpropylenediamine, N,N-dimethylpropylenediamine, N,N-diethylpropyllenediamine, or a combination thereof.
Embodiment 6 is the separation media of any one of embodiments 1 to 5, wherein the plurality of ligands comprise cation-exchange ligands comprising aminocarboxylic acid, aminosulfonic acid, or a combination thereof.
Embodiment 7 is the separation media of embodiment 6, wherein the cation-exchange ligand comprises aminobenzoic acid, aminodiacetic acid, aminopropanoic acid, 3-amino-1-propanesulfonic acid, 3-amino-1-ethylsulfonic acid, or a combination thereof, comprising a spacer between an amino group and an acid or sulfonate group that is 1 to 18 carbons, 1 to 10 carbons, 1 to 6 carbons, or 2 to 4 carbons long.
Embodiment 8 is the separation media of any one of embodiments 1 to 7, wherein the plurality of ligands comprises two or more of anion exchange ligands, cation exchange ligands, thiophilic ligands, hydrophilic ligands, and hydrophobic interaction ligands.
Embodiment 9 is the separation media of any one of embodiments 1 to 8, wherein the plurality of ligands comprises ligands with cation-exchange functionality and thiophilic functionality.
Embodiment 10 is the separation media of embodiment 9, wherein the plurality of ligands comprises mercaptobenzoic acid, mercaptosulfonic acid, a salt thereof, or a combination thereof, preferably wherein the plurality of ligands comprises sodium 3-mercapto-1-propanesulfonate.
Embodiment 11 is the separation media of embodiments 1 to 10, wherein the plurality of ligands are formed from a ligand having formula (I):

Fh-Sp-Sg (I)

wherein Fh is the functional handle, Sp is a spacer, and Sg is a functional separation group,

wherein the functional handle is selected from amines; alcohols; activated alcohols; epoxides; isocyanates; alkenes; alkynes; cycloalkenes; cyclooctynes; thiols; disulfides; azides; thioisocyanates; N-hydroxysuccinimide; maleimides; activated esters; and activated carboxylic acids,

wherein the spacer is a carbon chain of length C1 to C18, C1 to C10, C1 to C6, C1 to C4, C1 to C3, or C2 to C4, optionally substituted with one or more ethers, esters, benzyls, phenyls, or amides along the carbon chain, and

wherein the functional separation group is a cation-exchange separation group, anion-exchange separation group, hydrophobic separation group, or thiophilic separation group.

Embodiment 12 is the separation media of embodiment 11, wherein the functional separation group comprises an acid, a carboxylic acid, a sulfonic acid, a phosphoric acid, a carboxylate, a sulfonate, or a phosphate.
Embodiment 13 is the separation media of embodiment 11, wherein the functional separation group comprises a primary amine, a secondary amine, a tertiary amine, a quaternary amine, or a combination thereof, and optionally wherein the functional handle comprises a secondary amine, a tertiary amine, or a quaternary amine, and wherein adjacent amines are separated by an aliphatic carbon chain of 1 to 18 carbons, 1 to 10 carbons, 1 to 6 carbons, 1 to 4 carbons, or 2 to 4 carbons.
Embodiment 14 is the separation media of embodiment 11, wherein the functional separation group comprises an aliphatic chain with two carbons or longer (optionally butyl, pentyl, hexyl, heptyl, octyl, nonyl, decyl, undecyl, and dodecyl), benzyl, phenyl, phenol, pyridine, boronic acid, a branched polymer (optionally polypropylene glycol), a sulfur-containing thiophilic ligand (optionally propanethiol, 2-butanethiol, 3,6-dioxa-1,8-octanedithiol, octanethiol, benzyl mercaptan, 2-mercaptopyridine, thiophenol, 1,2-ethanedithiol, 1,4-benzenedimethanethiol, or 2-phenylethanethiol), or a combination thereof.
Embodiment 15 is the separation media of embodiment 11, wherein the functional handle comprises a thiol and the functional separation group comprises an acid, a sulfonic acid, or a phosphate.
Embodiment 16 is a separation device comprising: a housing; and separation media disposed within the housing, the separation media comprising: a membrane; and a plurality of ligands immobilized on the membrane, the plurality of ligands comprising anion-exchange ligands, cation-exchange ligands, thiophilic ligands, hydrophobic interaction ligands, hydrophilic ligands, or a combination thereof.
Embodiment 17 is the separation device of embodiment 16, wherein the housing comprises a cassette or a column.
Embodiment 18 is the separation device of embodiment 16 or 17, wherein the separation media is configured for separation of target molecules comprising nucleobases from a reaction mixture.
Embodiment 19 is the separation device of any one of embodiments 16 to 18, wherein the separation media is configured for use with organic solvents.
Embodiment 20 is the separation device of any one of embodiments 16 to 19, wherein the plurality of ligands comprise anion exchange ligands comprising an aliphatic diamine or triamine comprising 1 to 18 carbons between adjacent amines.
Embodiment 21 is the separation device of embodiment 20, wherein the anion exchange ligand comprises N,N-dimethylethylenediamine, N,N-dimethylpropylenediamine, N,N-dimethylpropylenediamine, N,N-diethylpropyllenediamine, or a combination thereof.
Embodiment 22 is the separation device of any one of embodiments 16 to 21, wherein the plurality of ligands comprise cation-exchange ligands comprising aminocarboxylic acid, aminosulfonic acid, or a combination thereof.
Embodiment 23 is the separation device of embodiment 22, wherein the cation-exchange ligand comprises aminobenzoic acid, aminodiacetic acid, aminopropanoic acid, 3-amino-1-propanesulfonic acid, 3-amino-1-ethylsulfonic acid, or a combination thereof, comprising a spacer between an amino group and an acid or sulfonate group that is 1 to 18 carbons, 1 to 10 carbons, 1 to 6 carbons, or 2 to 4 carbons long.
Embodiment 24 is the separation device of any one of embodiments 16 to 23, wherein the plurality of ligands comprises two or more of anion exchange ligands, cation exchange ligands, thiophilic ligands, hydrophilic ligands, and hydrophobic interaction ligands.
Embodiment 25 is the separation device of any one of embodiments 16 to 24, wherein the plurality of ligands comprises ligands with cation-exchange functionality and thiophilic functionality.
Embodiment 26 is the separation device of embodiment 25, wherein the plurality of ligands comprises mercaptobenzoic acid, mercaptosulfonic acid, a salt thereof, or a combination thereof, preferably wherein the plurality of ligands comprises sodium 3-mercapto−1-propanesulfonate.
Embodiment 27 is the separation device of embodiments 16 to 26, wherein the plurality of ligands are formed from a ligand having formula (I):

Fh-Sp-Sg (I)

wherein Fh is the functional handle, Sp is a spacer, and Sg is a functional separation group,

wherein the functional handle is selected from amines; alcohols; activated alcohols; epoxides; isocyanates; alkenes; alkynes; cycloalkenes; cyclooctynes; thiols; disulfides; azides; thioisocyanates; N-hydroxysuccinimide; maleimides; activated esters; and activated carboxylic acids,

wherein the spacer is a carbon chain of length C1 to C18, C1 to C10, C1 to C6, C1 to C4, C1 to C3, or C2 to C4, optionally substituted with one or more ethers, esters, benzyls, phenyls, or amides along the carbon chain, and

wherein the functional separation group is a cation-exchange separation group, anion-exchange separation group, hydrophobic separation group, or thiophilic separation group.

Embodiment 28 is the separation device of embodiment 27, wherein the functional separation group comprises an acid, a sulfonic acid, or a phosphate.
Embodiment 29 is the separation device of embodiment 27, wherein the functional separation group comprises a primary amine, a secondary amine, a tertiary amine, a quaternary amine, or a combination thereof, and optionally wherein the functional handle comprises a secondary amine, a tertiary amine, or a quaternary amine, and wherein adjacent amines are separated by an aliphatic carbon chain of 1 to 18 carbons, 1 to 10 carbons, 1 to 6 carbons, 1 to 4 carbons, or 2 to 4 carbons.
Embodiment 30 is the separation device of embodiment 27, wherein the functional separation group comprises an aliphatic chain with two carbons or longer (optionally butyl, pentyl, hexyl, heptyl, octyl, nonyl, decyl, undecyl, and dodecyl), benzyl, phenyl, phenol, pyridine, boronic acid, a branched polymer (optionally polypropylene glycol), a sulfur-containing thiophilic ligand (optionally propanethiol, 2-butanethiol, 3,6-dioxa-1,8-octanedithiol, octanethiol, benzyl mercaptan, 2-mercaptopyridine, thiophenol, 1,2-ethanedithiol, 1,4-benzenedimethanethiol, or 2-phenylethanethiol), or a combination thereof.
Embodiment 31 is the separation device of embodiment 27, wherein the functional handle comprises a thiol and the functional separation group comprises an acid, a carboxylic acid, a sulfonic acid, a phosphoric acid, a carboxylate, a sulfonate, or a phosphate.
Embodiment 32 is a method of purifying a target molecule, the method comprising: passing a solution comprising the target molecule through a membrane chromatography device, the target molecule comprising a nucleic acid, nucleotide, nucleoside, nucleobase, or an analogue or derivative thereof, and the membrane chromatography device comprising: a housing; and separation media disposed within the housing, the separation media comprising: a membrane; and a plurality of ligands immobilized on the membrane, the plurality of ligands comprising anion exchange ligands, cation exchange ligands, thiophilic ligands, hydrophobic interaction ligands, hydrophilic ligands, or a combination thereof.
Embodiment 33 is the method of embodiment 32, wherein the target molecule is purified from a solution comprising a reaction mixture after synthesis of the target molecule.
Embodiment 34 is the method of embodiment 32 or 33, wherein the separation media comprises an anion exchange membrane.
Embodiment 35 is the method of any one of embodiments 32 to 34, wherein residence time of the solution in the membrane chromatography device is 60 s or lower.
Embodiment 36 is the method of any one of embodiments 32 to 35, wherein the target molecule is a nucleotide or nucleic acid.
Embodiment 37 is the method of any one of embodiments 32 to 36, wherein the separation media comprises thiophilic cation-exchange membrane.
Embodiment 38 is the method of any one of embodiments 32 to 37, wherein the thiophilic cation-exchange membrane comprises cation-exchange ligands with thiophilic functional groups.
Embodiment 39 is the method of any one of embodiments 32 to 38, wherein the solution comprises an organic solvent.
Embodiment 40 is the method of any one of embodiments 32 to 39, wherein the plurality of ligands comprises ligands with cation-exchange functionality and thiophilic functionality.
Embodiment 41 is the method of any one of embodiments 32 to 40, wherein the plurality of ligands comprises mercaptobenzoic acid, mercaptosulfonic acid, a salt thereof, or a combination thereof, preferably wherein the plurality of ligands comprises sodium 3-mercapto−1-propanesulfonate.
Embodiment 42 is the method of any one of embodiments 32 to 41, wherein the separation media is according to any one of embodiments 1 to 15 and/or the separation device is according to any one of embodiments 16 to 31.

### EXAMPLES

These examples are merely for illustrative purposes only and are not meant to be limiting on the scope of the appended claims. All parts, percentages, ratios, etc. in the examples and the rest of the specification are by weight, unless noted otherwise.

The performance of various types of separation membranes was tested and evaluated against control samples.

### Test Methods

A dynamic binding capacity at 10% breakthrough (DBC_{10%}) can be determined via a standard chromatography method, e.g., using Cytiva AKTA pure Fast Protein Liquid chromatography (FPLC). First, the said separation media is packed into a housing unit. Then, the contained separation media is connected to FPLC. Next, feed material is passed though the separation media under certain column volumes per minute flowrate (CV/min) until the effluent concentration of the target reaches 10% of the feed concentration, as determined by UV signals at suitable wavelengths. At the end, based on the holdup volume in the FPLC system and separation media volume, the DBC_{10%} is calculated as follows:

((Volume to 10% breakthrough-holdup volume)×(feed concentration))/(volume of separation media)=DBC_{10%} expresses as mg target material/mL chromatography media.

### Sample Testing

DBC_{10%} was determined using chromatography as described above. A complete bind-and-elute chromatograph typically contains four steps:

Step 1—Equilibration: the contained separation media is equilibrated with buffer A.

Step 2—Loading: loading material is injected/pumped through separation media until 10% breakthrough is attained.

Step 3—Washing: the media is washed using a wash buffer. The wash buffer can be a single buffer or multiple buffers to wash away some of the impurities. The wash buffer can include buffer A or a different buffer B, or a combination of buffers comprising of buffers A and/or B and/or additional buffers (C, D, E, F, etc.), or a gradient transitioning between buffers A and/or B and/or additional buffers (C, D, E, F, etc.), or a gradient transitioning between combinations of buffers A and/or B and/or additional buffers (C, D, E, F, etc.).

Step 4—Elution: buffer C is used to elute loaded material (such as target and some of the impurities) from the separation media. The elution buffer can include buffer C or a different buffer B, or a combination of buffers comprising of buffers C and/or B and/or additional buffers (A, D, E, F, etc.), or a gradient transitioning between buffers C and/or B and/or additional buffers (A, D, E, F, etc.), or a gradient transitioning between combinations of buffers C and/or B and/or additional buffers (A, D, E, F, etc.).

The eluate can be collected for further analysis.

In addition to the four major steps mentioned above, stripping and/or clean-in-place (CIP) steps may also be performed in some cases prior to restarting the cycle.

### Example 1— Preparation of AEX Membranes

A regenerated cellulose membrane having a diameter of 47 mm and a thickness of 70 μm was soaked at 40° C. for 16 hours in a solution of 300 mg of N,N-disuccimidylcarbonate (DSC) and 139 μL of triethylamine (TEA) dissolved in 10 mL of dimethylsulfoxide (DMSO). The membrane was subsequently placed in a solution of 100 μL N,N-dimethylethylenediamine (DMEDA) for every mL DMSO at room temperature for 16 hours. Finally, the membrane was placed in 1 M Tris pH 8.0, for 16 hours.

### Example 2—Preparation of Thiophilic-CEX Membranes

A regenerated cellulose membrane having a diameter of 47 mm and a thickness of 70 μm was soaked at 40° C. for 16 hours in a solution of 300 mg of DSC and 139 μL of TEA for 16 hours. The membrane was soaked at 40° C. for 16 hours in a solution of 300 mg of sodium 3-mercaptopropanesulfonate and 0.5 mL of TEA dissolved in 10 mL of DMSO. Finally, the membrane was placed in 200 mM Tris pH 8.0, for 16 hours.

### Example 3—DBC of Separation Media

The dynamic binding capacity of a thiophilic-CEX separation media was tested at various flowrates. The separation membrane was prepared according to Example 2.

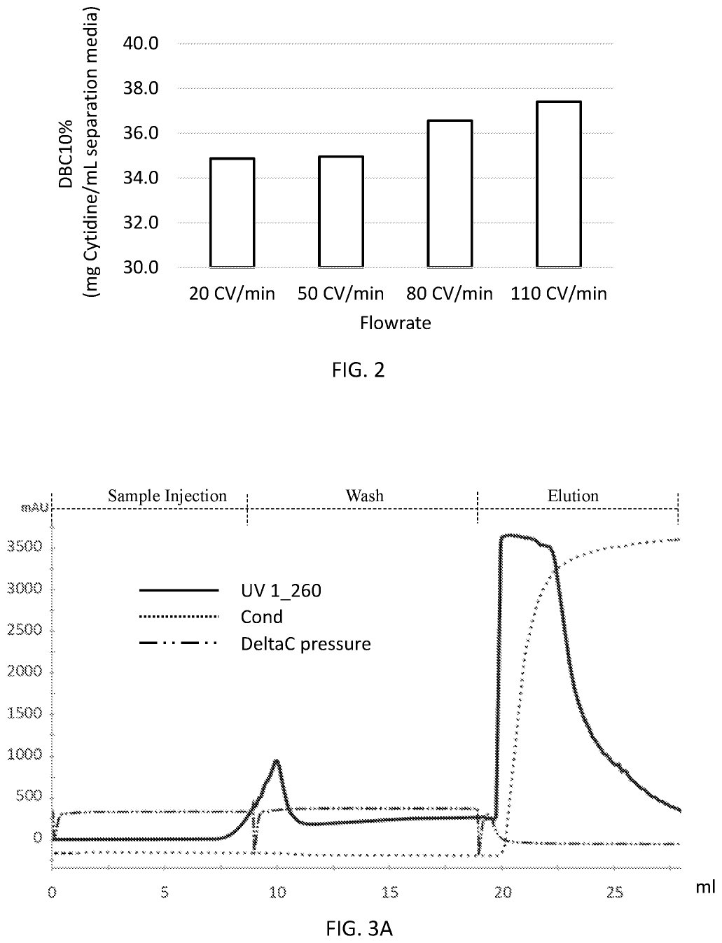

Four layers of 24 mm circular separation membranes were packed into a mini column (membrane volume=0.1 mL) and connected to Cytiva AKTA Pure to determine dynamic binding capacity. In this example, the loading material was 0.8 mg/mL cytidine in 10 mM phosphoric acid pH 2.0. The cytidine solution was applied to the membranes at various flowrates as identified by column volumes per minute (CV/min) until 10% breakthrough. The DBC_{10%} results are presented graphically are shown in FIG. 2.

It can be seen from FIG. 2 that increasing the flowrate increases the DBC_{10%} of cytidine. The trans-column pressure (delta C) pressure was 0.06 MPa with 110 CV/min, which indicates that a further increase of flowrate may be possible.

### Example 4—Separation of Cytidine with Organic Buffer System

The ability of the thiophilic-CEX separation media to separate cytidine in an organic buffer system was tested. The separation membrane was prepared according to Example 2. Two commercially available products, HITRAP® SP HP cation exchange chromatography column available from Cytiva in Marlborough, Mass., and a NATRIFLO® HD-Sb column available from Natrix Separations were used as comparative samples. HITRAP® SP HP is a resin-based strong cation exchange chromatography column product and NATRIFLO® HD-Sb is a hydrogel-based strong cation exchange column product augmented with hydrophobic interaction groups.

Eight layers of 24 mm circular separation membranes were packed into a polypropylene column housing (membrane volume=0.2 mL) and connected to Cytiva AKTA Pure for a bind-and-elute chromatographic separation process. Cytidine was loaded under a determined column volume per minute flowrate (CV/min) until 10% breakthrough. The buffer list shown in TABLE 1 was used in the separation process.

TABLE 1
Buffer List.

| Step | Buffer |
|---|---|
| Equilibration | 5 mM phosphoric acid pH 2.0, 50% EtOH in water |
| Column Wash | 50% EtOH in water |
| Elution | 0.5M NaCl pH 7, 50% EtOH |

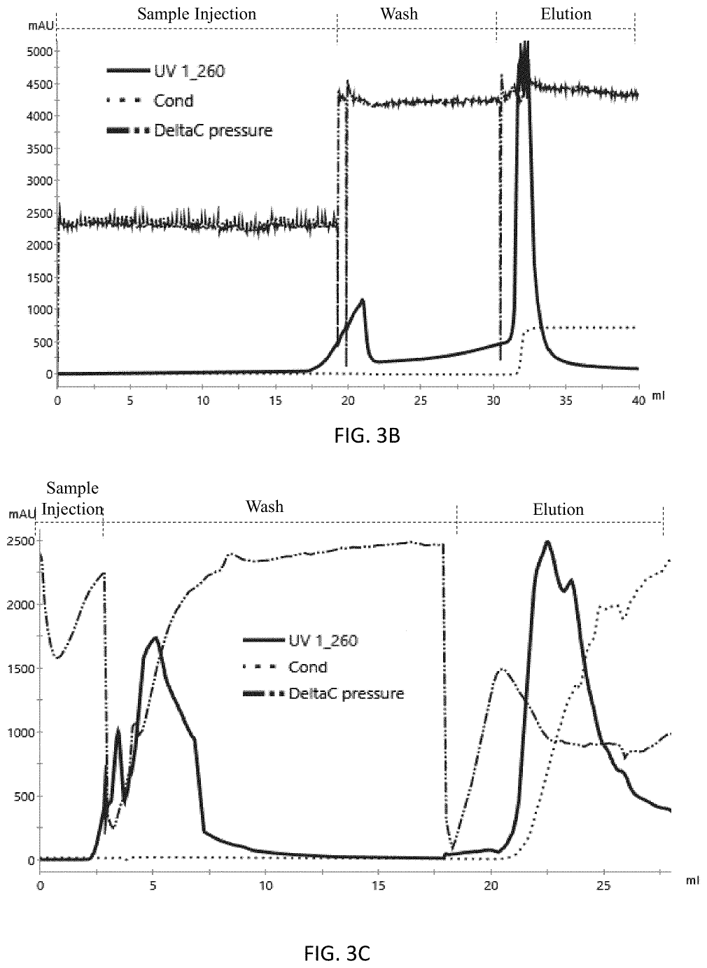

Chromatograms of the sample separation membrane, resin (HITRAP® SP HP), and hydrogel (NATRIX® HD-Sb) commercial products are shown in FIGS. 3A, 3B, and 3C, respectively. The operational parameters and outputs, including flowrate, DBC_{10%} and transcolumn pressure (delta C) are summarized in TABLE 2.

TABLE 2
Operational parameters.

| | | DBC 10% | |
|---|---|---|---|
| | | (mg cytidine/mL | |
| | Flowrate | chromatography | Highest delta C |
| Sample | (CV/min) | media) | pressure (MPa) |
| Thiophilic-CEX | 10 | 35.1 | 0.07 |
| Membrane | | | |
| HITRAP ® SP HP | 0.5 | 17 | 0.07 |
| NATRIFLO ® HD-Sb | 12.5 | 1.9 | 0.09 |

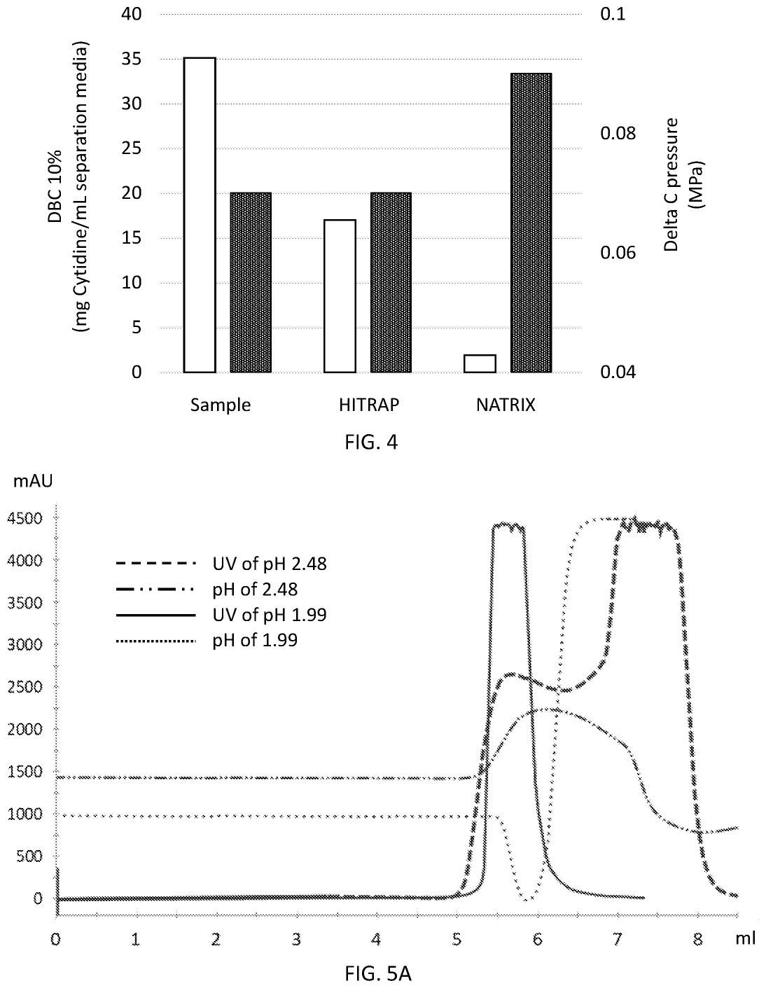

The binding capacities of the columns are compared in FIG. 4. It was observed that the separation membrane has a binding capacity that is about 2 times of the binding capacity of the resin and about 18 times of binding capacity of the hydrogel using the organic buffer system of TABLE 1. At the same transcolumn pressure, the flowrate of the separation media is at least 20 times faster than that of resin.

### Example 5—Separation of Cytidine with Aqueous Buffer System

The ability of CEX separation resin to separate cytidine in an aqueous buffer system was tested. The separation resin was HITRAP® SP HP resin purchased from Cytiva.

A one mL HITRAP® SP HP resin was connected to Cytiva AKTA Pure for a bind-and-elute chromatographic separation process in a fully aqueous system (20 mM Sodium Phosphate with various pHs ranging from 2.48 to 1.99).

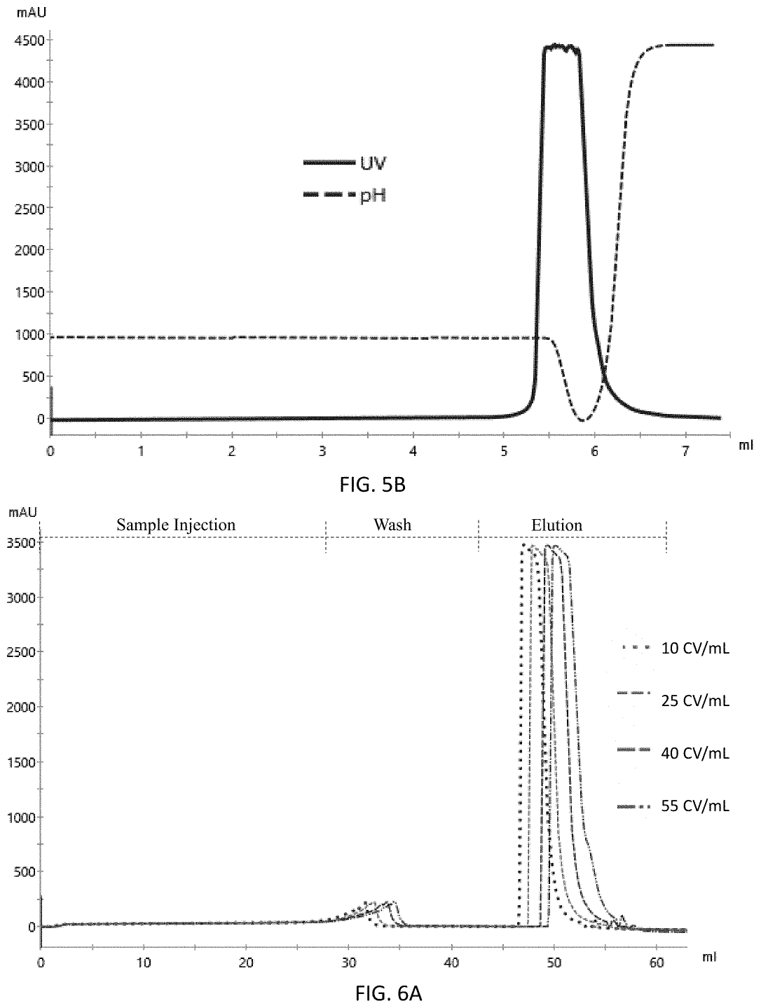

The bind-and-elute profiles of cytidine dissolved in 20 mM sodium phosphate in either a solution with pH of 2.48 or a pH of 1.99 are shown in FIG. 5A.

Initial cycle with loading at pH 2.48 resulted in a broadened bimodal elution peak commencing in the tail-end of the wash phase. The bind-and-elute profile from figure BB using 20 mM Sodium Phosphate pH 1.99 is shown in FIG. 5B.

It was observed that decreasing the loading pH to 1.99 from 2.48 was able to resolve elution into a single sharp elution peak compared to the broad bimodal peak observed in bind and elute cycles at pH 2.48. It is hypothesized that protonated amines on cytidine are more prevalent in an acidic environment (reduced pH), which allowed for stronger adsorption to the CEX column over deprotonated cytidine present in a more elevated pH. While purification was able to be performed using a resin, it was done at a significantly slower flowrate and thus results in low chromatographic productivity for the overall process compared to membranes with the same ligand.

### Example 6—DBC and Recovery of AEX

Anion-exchange (AEX) separation media with a pore size of 1 μm was prepared according to Example 1. Eight layers of 24 mm circular AEX membrane were packed into housing unit (membrane volume=0.2 mL) and connected to Cytiva AKTA pure to determine dynamic binding capacity. In this example, the loading material was 0.25 mg/mL adenosine-5′-monophosphate (AMP, 96.98%) dissolved in equilibration buffer. AMP was loaded under different column volumes per minute flowrate (CV/min) until 10% breakthrough was reached. The list of buffers used in the separation process is shown in TABLE 3.

TABLE 3
Buffer List.

| Step | Buffer |
|---|---|
| Equilibration | 20 mM Tris pH 7 |
| Column Wash | DI water |
| Elution | 1M NaCl |

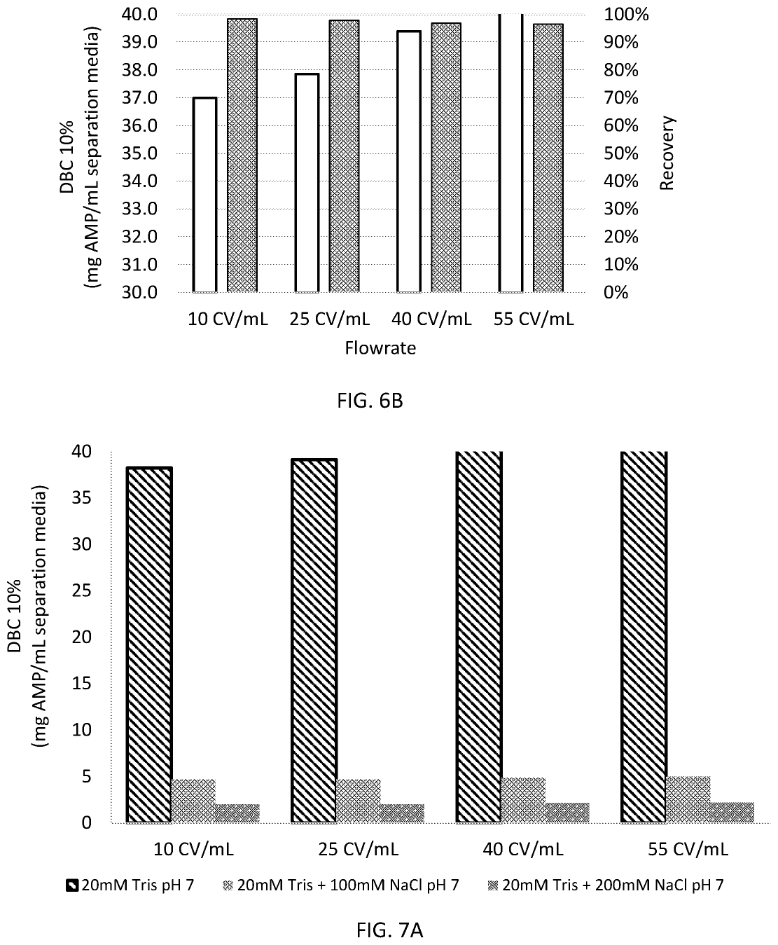

Chromatogram overlays are shown in FIG. 6A. DBC_{10%} of AEX separation media and AMP recovery are shown in FIG. 6B. It can be seen from FIG. 6B that increasing the flowrate does not decrease the binding capacity of said AEX separation media or AMP recovery.

### Example 7—DBC of AEX in Buffer Conditions with Different Conductivities

Eight layers of 24 mm circular AEX membranes were packed into a housing unit (membrane volume=0.2 mL) and connected to Cytiva AKTA pure to determine dynamic binding capacity. The membrane was prepared according to Example 1. In this example, the feed was 0.25 mg/mL AMP in equilibration buffers with 0, 100 mM, or 200 mM NaCl added. AMP was loaded under different column volume per minute flowrate (CV/min) until 10% breakthrough and the DBCs are shown in FIG. 3. The list of buffers used in the separation process is shown in TABLE 4.

TABLE 4
Buffer List

| Step | Buffers |
|---|---|
| Equilibration | 20 mM Tris pH 7 (with 0, 100 mM, or 200 mM NaCl) |
| Column Wash | DI water |
| Elution | 1M NaCl |

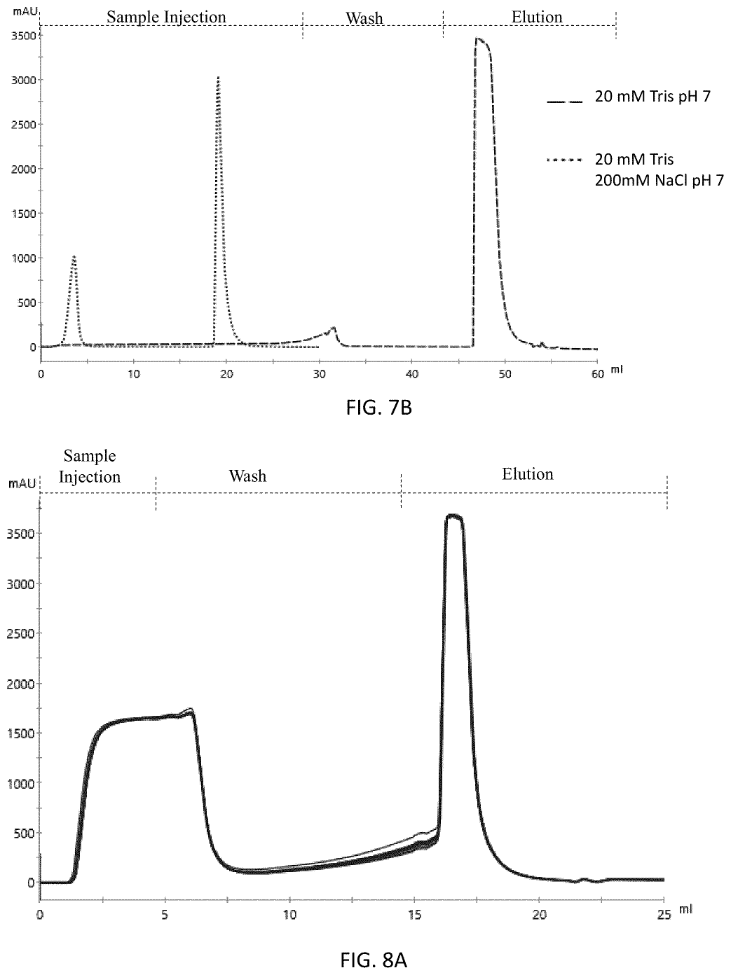

DBC_{10%} under different flowrates and salt conditions are shown in FIG. 7A. Chromatogram overlays are shown in FIG. 7B. It can be seen from FIG. 3 that in each buffer condition, increasing the flowrate does not decrease the binding capacity of said AEX separation media. However, as shown in FIG. 7B, the increased salt concentration decreases binding capacity of said AEX separation media significantly. At 10 CV/mL, the DBC decreases from 38 mg/mL without addition of salt to 2 mg/mL with addition of 200 mM NaCl.

### Example 8—Separation of Difluoro-Substituted Nucleoside Derivative Intermediates with Various Alcohol and Amine Protections in an Organic Buffer System

The ability of thiophilic-CEX separation media to separate out difluoro-substituted nucleoside derivative intermediates with deprotected amines from a mixture containing difluoro-substituted nucleoside derivatives with combinations of mono-, and/or di-protected alcohols and/or protected amine and excess protecting agent in organic solvent (diluted 50× by equilibration buffer listed in Table 3) was tested. The separation membrane was prepared according to Example 2.

Eight layers of 24 mm circular separation membranes were packed into a membrane housing (membrane volume=0.2 mL) and connected to Cytiva AKTA Pure for a bind-and-elute chromatographic separation process. A sample containing a mixture of difluoro-substituted nucleoside derivatives with combinations of mono-, and/or di-protected alcohols and/or protected amine and excess protecting agent in organic solvent (diluted 50× by equilibration buffer listed in Table 3) was loaded under a determined column volume per minute flowrate (CV/min) until 10% breakthrough was reached. The buffers listed in TABLE 5 were used in the separation process.

TABLE 5
Buffer List.
Buffer

5 mM phosphoric acid pH 2/50% EtOH in water
50% EtOH
0.5M NaCl pH 7/50% EtOH in water
30% isopropyl alcohol

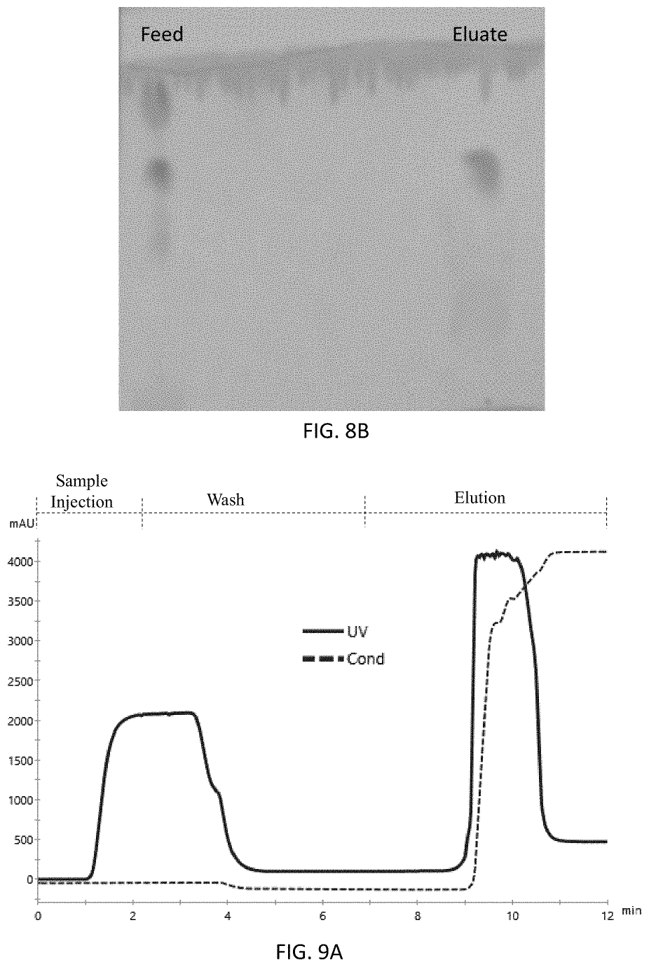

This process was repeated 10 time with the respective chromatograms overlaid in FIG. 8A.

Bind-and-elute purification cycles were found to be consistent run to run. Samples were taken every 5^{th} run and concentrations were assessed via UV absorbance using NANODROP™ Lite UV-Vis Spectrophotometer (available from Thermo Scientific) at 260 nm. Yield was found to be consistent with a Coefficient of Variance of 5.17%.

Sample of feed and eluate were also taken for thin layer chromatography (TLC) analysis. A photograph of the TLC plate is shown in FIG. 8B, with the feed sample on the left and eluate sample on the right. As indicated in FIG. 8B, the reduction of the 3 species in crude feed (left) representing difluoro-substituted nucleoside derivatives with mono-alcohol protection (bottom), di-alcohol protection (middle), and tri- (top) protection to a single band of enriched di-protected species in the eluate (right) at the same position with di-protected species band in feed suggests successful exploitation of the amine pendent group on the pyrimidine for CEX purification in a partially organic solvent system. Tri-protected species here refers to a difluoro-substituted nucleoside derivative with di-protected alcohols and a protected amine. It is hypothesized that protonated amine presents only in any combination un-, mono-, and di-protected alcohols in any combination without amine protection allowed for stronger attraction to the CEX column over other variants with protected amines. For molecules with amine protections, the pendent amine is unable to be protonated and therefore exhibits much weaker adsorption to the column via CEX mechanisms.

### Example 9—Comparative Study

Separation of difluoro-substituted nucleoside derivative intermediates with deprotected amines from a mixture containing difluoro-substituted nucleoside derivatives with combinations of mono-, and/or di-protected alcohols and/or protected amine and excess protecting agent in organic solvent (diluted 50× by equilibration buffer listed in Table 3) was tested using a cation exchange membrane prepared according to Example 2 and a HITRAP® SP HP cation exchange resin column were tested.

Tests were performed at a flowrate of 1 mL/min and the process time was 12 minutes. The solution was diluted in a low conductivity ethanol/phosphate mixture (low conductivity) and the columns were eluted with 1 M NaCl (high conductivity).

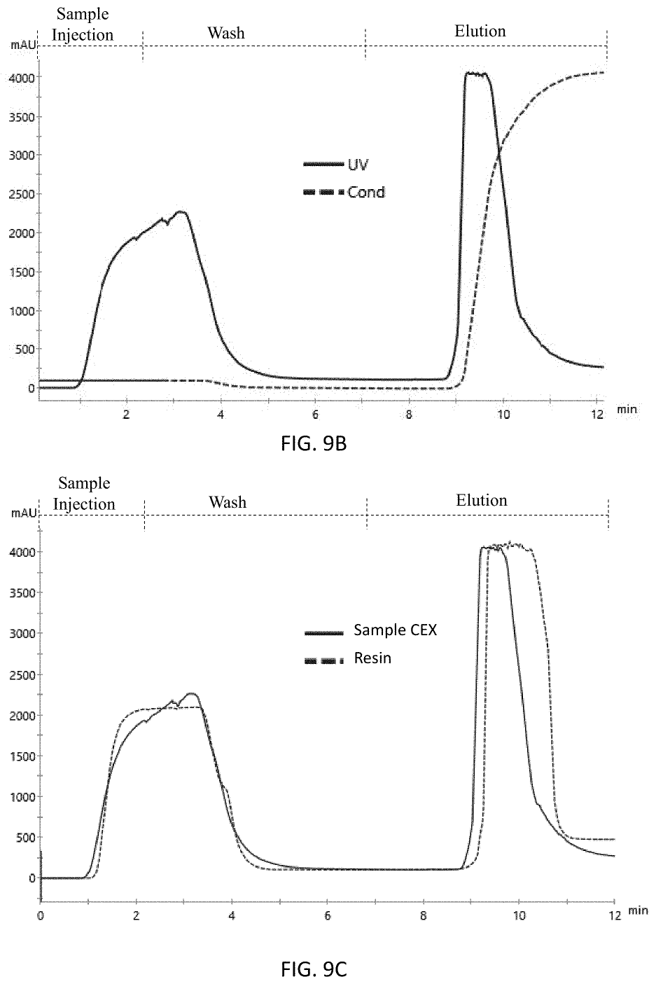

The resin column had a 1 mL resin volume and the estimated binding capacity was 8 mg/mL. The projected large scale residence time is 120 min or longer per cycle. The bind-and-elute data is shown in FIG. 9A.

The membrane column had a 0.1 mL media volume and the estimated binding capacity was 80 mg/mL. The projected large scale residence time is about 24 min per cycle. The bind-and-elute data is shown in FIG. 9B.

The bind-and-elute data from both columns is overlaid in FIG. 9C

While purification was able to be performed using a resin, it was done at a significantly slower flowrate and thus results in low chromatographic productivity for the overall process compared to separations performed with membranes.

Comparing purity via HPLC, a slightly higher purity eluate was obtained via purifications employing membrane over that obtained while employing resins. HPLC chromatograms exhibiting purity of elution pools of purifications performed with resin vs membrane are shown in FIG. 9D. Additionally, rotoevaporation of the eluate was observed to result in crystals of targeted difluoro-substituted nucleoside derivatives with di-protected alcohols and unprotected amines from feed containing organic solvent and protecting agents in excess. Rotoevaporation of unpurified feed containing organic solvent and protecting agents in excess does not result in crystalline product, indicating separation of desired targeted difluoro-substituted nucleoside derivatives with unprotected amines from other constituents.

Elutions could be performed by increasing organic solvent composition in conjunction with increasing salt concentration. This is particularly advantageous for purifications prior to downstream desiccation or subsequent synthetic steps performed in low water conditions. The use of organic solvent allows for a greater percentage of solvent to be more easily removed and recovered, potentially accelerating rotoevaportory steps. Furthermore, use of less salt adds less materials that need be removed in subsequent purification steps. Elutions were performed successfully with as low as 5% 1 M NaCl buffer in 95% ethanol (50 mM final concentration).

### Example 10—DBC of MCP

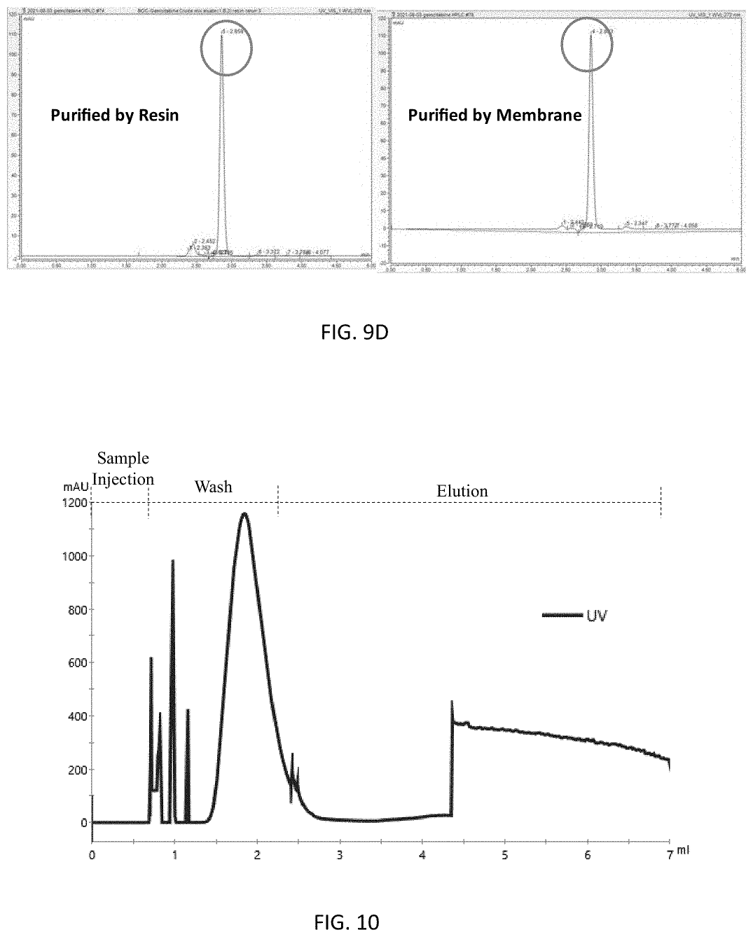

The ability of a resin based mercaptopyridine (MCP) (Cytiva PlasmidSelect) to separate cytidine in an aqueous buffer system was tested. Cytidine at a concentration of 0.3125 mg/mL in 20 mM sodium acetate 3 M ammonium sulfate pH 4.1 was applied to a resin based MCP (Cytiva PlasmidSelect) column to assess binding with elution in 20 mM Tris pH 7.0.
A successful bind and elute cycle was performed, an elution peak was observed as depicted in FIG. 10. It is expected that increased productivity by virtue of decreased purification time would be the result of a similar purification using a membrane-based MCP. Furthermore, it is hypothesized that these and similar conditions would also be applicable to other HIC columns.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A separation media comprising:
a membrane; and
a plurality of ligands immobilized on the membrane, the plurality of ligands comprising anion-exchange ligands, cation-exchange ligands, thiophilic ligands, hydrophobic interaction ligands, hydrophilic ligands, or a combination thereof.

2. The separation media of claim 1, wherein the separation media is configured for separation of target molecules comprising nucleotides, nucleosides, nucleobases, their derivatives and analogues, and combinations thereof, from a reaction mixture.

3. The separation media of claim 1, wherein the separation media is configured for use with organic solvents.

4. The separation media of claim 1, wherein the plurality of ligands comprise anion-exchange ligands comprising an aliphatic diamine or triamine comprising 1 to 18 carbons between adjacent amines.

5. The separation media of claim 4, wherein the anion exchange ligand comprises N,N-dimethylethylenediamine, N,N-dimethylpropylenediamine, N,N-dimethylpropylenediamine, N,N-diethylpropyllenediamine, or a combination thereof.

6. The separation media of claim 1, wherein the plurality of ligands comprise cation-exchange ligands comprising aminocarboxylic acid, aminosulfonic acid, or a combination thereof.

7. The separation media of claim 6, wherein the cation-exchange ligand comprises aminobenzoic acid, aminodiacetic acid, aminopropanoic acid, 3-amino-1-propanesulfonic acid, 3-amino-1-ethylsulfonic acid, or a combination thereof, comprising a spacer between an amino group and an acid or sulfonate group that is 1 to 18 carbons, 1 to 10 carbons, 1 to 6 carbons, or 2 to 4 carbons long.

8. The separation media of claim 1, wherein the plurality of ligands comprises two or more of anion exchange ligands, cation exchange ligands, thiophilic ligands, hydrophilic ligands, and hydrophobic interaction ligands.

9. The separation media of claim 1, wherein the plurality of ligands comprises ligands with cation-exchange functionality and thiophilic functionality.

10. The separation media of claim 9, wherein the plurality of ligands comprises mercaptobenzoic acid, mercaptosulfonic acid, a salt thereof, or a combination thereof, preferably wherein the plurality of ligands comprises sodium 3-mercapto−1-propanesulfonate.

11. A separation device comprising:
a housing; and
separation media disposed within the housing, the separation media comprising:
a membrane; and
a plurality of ligands immobilized on the membrane, the plurality of ligands comprising anion-exchange ligands, cation-exchange ligands, thiophilic ligands, hydrophilic ligands, hydrophobic interaction ligands, or a combination thereof.

12. The separation device of claim 11, wherein the housing comprises a cassette or a column.

13. The separation device of claim 11, wherein the separation media is configured for separation of target molecules comprising nucleobases from a reaction mixture.

14. The separation device of claim 11, wherein the separation media is configured for use with organic solvents.

15. The separation device of claim 11, wherein the plurality of ligands comprise anion exchange ligands comprising an aliphatic diamine or triamine comprising 1 to 18 carbons between adjacent amines.

16. The separation device of claim 15, wherein the anion exchange ligand comprises N,N-dimethylethylenediamine, N,N-dimethylpropylenediamine, N,N-dimethylpropylenediamine, N,N-diethylpropyllenediamine, or a combination thereof.

17. The separation device of claim 11, wherein the plurality of ligands comprise cation-exchange ligands comprising aminocarboxylic acid, aminosulfonic acid, or a combination thereof.

18. The separation device of claim 17, wherein the cation-exchange ligand comprises aminobenzoic acid, aminodiacetic acid, aminopropanoic acid, 3-amino-1-propanesulfonic acid, 3-amino-1-ethylsulfonic add, or a combination thereof, comprising a spacer between an amino group and an acid or sulfonate group that is 1 to 18 carbons, 1 to 10 carbons, 1 to 6 carbons, or 2 to 4 carbons long.

19. The separation device of claim 11, wherein the plurality of ligands comprises two or more of anion exchange ligands, cation exchange ligands, thiophilic ligands, hydrophilic ligands, and hydrophobic interaction ligands.

20. The separation device of claim 11, wherein the plurality of ligands comprises ligands with cation-exchange functionality and thiophilic functionality.

21. The separation device of claim 20, wherein the plurality of ligands comprises mercaptobenzoic acid, mercaptosulfonic acid, a salt thereof, or a combination thereof, preferably wherein the plurality of ligands comprises sodium 3-mercapto-1-propanesulfonate.

22-32. (canceled)
